(19) United States
(12) Patent Application Publication
(10) Pub. No.: US 2007/0226007 A1
(43) Pub. Date: Sep. 27, 2007

(54) Health service support system

(76) Inventors: Takanobu Osaki, Nishitokyo (JP); Hideyuki Ban, Kodaira (JP); Hiromi Kurisaki, Matsudo (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/492,915

(22) Filed: Jul. 26, 2006

(30) Foreign Application Priority Data
Mar. 27, 2006 (JP) 2005-084462

(51) Int. Cl. G06Q 10/00; A61B 5/00

(52) U.S. Cl. 705/2; 600/300

(57) ABSTRACT

A method for deciding items and parameters for selection of health guidance receivers, and a method for readjusting the method. To that end, there is provided a health service support system including a routine for computing a health guidance effect on a time-series basis by the category obtained by combining the items and dividing value candidate with the items and dividing value candidate on the basis of the health checkup data units, the health guidance data units, and the medical fee receipt data units, a routine for computing a feature quantity, in the form of an approximate straight line, from the time-series health guidance effect, a routine for selecting at least one item and parameter from the items and dividing value candidates, on the basis of the feature quantity, and a routine for creating a health guidance model associated with the order of priority in health guidance, using the items and parameters, as selected.

### CLAIM OF PRIORITY

The present application claims priority from Japanese application JP 2006-084462 filed on Mar. 27, 2006, the content of which is hereby incorporated by reference into this application.

### FIELD OF THE INVENTION

The invention relates to a system for planning and revamping health service in order to support the health service to be undertaken by a health insurer.

### BACKGROUND OF THE INVENTION

A health insurer provides the insured with health checkup and health guidance with the aim of stabilizing management of health service provided by the health insurer by optimizing health promotion of the insured and medical care costs. Health promotion, in particular, serves as an important occasion when the insured as an individual is caused to become aware of necessity of improving living and health conditions in light of results of health checkup. The health service including the health guidance and so forth have so far decided predetermined criteria, and the health service have been given to persons meeting the predetermined criteria, for example, persons who are determined as those requiring improvement in their living conditions as a result of health checkup. In reality, however, as there is limitation to the number of persons to whom health nurses can give guidance, so this is not a case where all the persons in need of health guidance are given the health guidance. Hence, there is a demand for a system for supporting management of the health service to enable the health insurer to effectively and efficiently manage the health service.

As a method for supporting the management by the health insurer, there are disclosed a number of methods as follows. In a medical care cost forecast system (JP 2005-50380 A), there is disclosed a method for forecasting future medical care costs as required by an organization from characteristics of persons belonging to the organization such as age, living habits, and so forth, data showing correlation between age and incidence, and database of medical care costs. Further, in a method for planning health service (health guidance) based on health indexes (JP 2004-341611 A), there is disclosed a method whereby decision is made on targets for intervention such as guidance in view of health levels and effects on medical care costs, thereby making assessment of effects of the intervention.

### SUMMARY OF THE INVENTION

An effect of health service including health guidance and so forth is considered to vary according to characteristics (health conditions, background factor) of a recipient of the health service, and so forth. For this reason, importance should be attached to items and parameters for selection of health guidance receivers, such as health checkup items, required for determining which characteristics to be looked at in selection of persons qualified for health guidance on a priority basis, and parameters for such determination, however, no consideration has so far been given to a method for deciding those items and parameters, enabling a higher effect of the health service to be obtained. No consideration has so far been given either to a method for making assessment of the effect of health guidance after taking a measure based on a plan such as the items and parameters as selected, and so forth, to thereby readjust the plan (the items and parameters as selected, and so forth). For example, in the case of giving health guidance to a category having a specific characteristic on the basis of a plan for giving guidance on a priority basis, it is deemed that an effect of health guidance will become unobtainable, or the category will decrease in size due to the effect of the health guidance, so that the characteristic of the category receiving the guidance and the effect of the health guidance will undergo a change with the elapse of time, however, in conventional examples described as above, no consideration has been given to a method for readjusting the plan according to such changes over time.

In order to resolve those problems, the invention provides a health service support system including a database having a health checkup data management routine 121 for storing health checkup data units of persons; a health guidance data management routine 122 for storing health guidance data units of the persons; and a medical fee receipt data management routine 123 for storing medical fee receipt data units including data units on medical care costs of the respective persons; a routine 106 for setting not less than two items and dividing value candidates combining health checkup data items for creation of a health guidance model with at least not less than two items and parameters having parameters for layering of the health checkup data items; a data interface for getting the health checkup data units of the persons, the health guidance data units of the persons, and the medical fee receipt data units, corresponding the items and dividing value candidates, respectively, from the health checkup data management routine, the health guidance data management routine, and the medical fee receipt data management routine, respectively; a routine for computing a health guidance effect on a time-series basis by the category obtained by combining the items and dividing value candidate with the items and dividing value candidate on the basis of the health checkup data units of the persons, the health guidance data units of the persons, and the medical fee receipt data units; a routine for computing a feature quantity, in the form of an approximate straight line, from the time-series health guidance effect; a routine for selecting at least one item and parameter from the items and dividing value candidates, on the basis of the feature quantity; and a routine for creating a health guidance model associated with the order of priority in health guidance, using the items and parameters, as selected.

The health guidance effect preferably represents a medical care cost cutback effect per person in the case of guiding persons belonging to a category obtained by combining the items and dividing value candidates with each other, and the medical care cost cutback effect preferably corresponds to an amount obtained when the persons belonging to the category obtained by combining the items and dividing value candidates with each other are divided into those having received guidance and those having received no guidance, and a medical care cost per person for those having received guidance is subtracted from a medical care cost per person for those having received no guidance.

The health service support system according to the invention may further comprise a routine for displaying the health guidance effect and the feature quantity, wherein the routine for selecting the at least one item and parameter from the items and dividing value candidates, on the basis of the feature quantity, selects the at least one item and parameter against the health guidance effect and the feature quantity, as displayed.

The routine for selecting the at least one item and parameter preferably selects an item and parameter where a widening gap between the approximate straight lines corresponding the effect to the category having the maximum effect, and the effect of the category having the minimum effect, respectively, among the feature quantity for the at least one item and parameter, is the largest against a plurality of categories obtained by combining the items and dividing value candidates with each other.

If a health guidance model previously created exists, the routine for setting the items and dividing value candidates preferably adopts items and parameters used by the health guidance model previously created as one of the items and dividing value candidates, and the routine for displaying the health guidance effect and the feature quantity preferably compares an health guidance effect in the case of using the items and parameters previously selected with an health guidance effect in the case of using the items and dividing value candidate newly adopted before displaying.

The routine for computing the health guidance effect on the time-series basis preferably make use of the health checkup data and the health guidance data during a time period “a”, and medical fee receipt data with the elapse of a time period “b” from the time period “a”, and preferably computes a difference in the medical care cost between the time period “a” and a time period “c” due to existence or nonexistence of the guidance executed during the time period “a” as the guidance effect by the category obtained by combining items and parameters, associated with the health checkup data during the time period “a”, a change of the guidance effect, on the time-series basis, being found by repeating computation by shifting the time period “a” and the time period “c” by the time period “a” every time the computation is made.

The health service support system according to the invention may further comprise an assessment and readjustment routine for computing an actual medical care cost on the basis of the health checkup data, the health guidance data, the medical fee receipt data, and the health guidance model, a forecast medical care cost on the basis of the health checkup data for a time period when the guidance is executed, and the health guidance model, and a planned medical care cost on the basis of the health checkup data when the health guidance model is created, and the health guidance model to be thereby compared with each other before displaying.

The routine for creating the health guidance model may create a plurality of health guidance models, and the assessment and readjustment routine may compute the planned medical care costs from the plurality of the health guidance models, respectively, so as to be displayed together with the actual medical care cost, and the forecast medical care cost.

Thus, with the health service support system according to the invention, the routine for computing the health guidance effect on the time-series basis finds the guidance effect and transition thereof by the category combining the items and parameters including age, sex, health checkup results, lifestyles, and so forth, obtained from health checkup data, and so forth, on the basis of the health checkup data units, health guidance data units, and the medical fee receipt data units, in the past. By so doing, it is possible to obtain an effect of selecting categorizing parameters for extracting the persons who should receive the health guidance in order to obtain a higher health guidance effect. If the persons who should receive the health guidance are selected particularly by selection of categorizing parameters on which a difference between the health guidance effect at the maximum, and the health guidance effect at the minimum is greater, this will enable a sharper distinction to be made between persons having a high health guidance effect, and persons having a low health guidance effect.

Further, if the persons who should receive the health guidance are selected by adoption of categorizing parameters on which the health guidance effect continues to rise with time due to transition of the health guidance effect on the category-by-category basis, this will have an effect of enabling persons who can gain a high health guidance effect to be selected as the persons who should receive the health guidance.

Still further, if the persons who should receive the health guidance are selected by adoption of categorizing parameters on which a difference in the health guidance effect between those having a high health guidance effect, and those having a low health guidance effect is widened over time, this will enable a sharper distinction to be made between those having a high health guidance effect, and those having a low health guidance effect, thereby enabling persons who can gain a high health guidance effect to be selected as the persons who should receive the health guidance. Accordingly, the health guidance plan adopting the categorizing parameters selected this way as the condition for selecting the persons who should receive the health guidance is effective in obtaining a higher health guidance effect.

Yet further, with the health service support system according to the invention, the items and parameters used in the health guidance model of the preceding year is compared with items and parameters newly adopted as a candidate. Hence, the system has an advantageous effect in that it is possible to compare the items and parameters used in the health guidance model of the preceding year with the candidate items and parameters newly adopted as the candidate at the time of readjustment of the health guidance plan, thereby selecting which of those items and parameters are preferable as parameters for selection of the persons who should receive the health guidance.

Further, with the health service support system according to the invention, the assessment and readjustment routine displays comparison among the planned medical care cost estimated at the time of planning, the forecast medical care cost as forecast after the health guidance is provided, and the actual medical care cost as found one year later, so that the user can make assessment of adequacy of the health guidance model used in the health guidance, adequacy of parameters adopted at the time of creating the model, and adequacy of the content of the health guidance. For example, if the user finds a discrepancy between the planned medical care cost and the forecast medical care cost, this will enable the user to become aware of a difference in tendency existing between the health guidance receivers at the time of planning and those at the time of executing the health guidance. Further, if the user finds a discrepancy between the forecast medical care cost and the actual medical care cost, this will enable the user to become aware of a possibility that a health guidance effect assumed at the time of planning has not been obtained. Because the user can check whether or not the health guidance plan is adequate by checking discrepancies among those medical care costs, the system has an advantageous effect in that the health guidance plan can be readjusted to thereby alter the items and divided value selection for the health guidance receivers to one capable of obtaining a higher health guidance effect.

Still further, with the health service support system according to the invention, in the routine for creating the health guidance model, health guidance receiver selection parameters under which a sharper distinction can be made between the persons having a high health guidance effect, and the persons having a low health guidance effect can be obtained on the basis of time series transition of the health guidance effect, and further, in the assessment and readjustment routine, the health guidance effects for the health insurer in whole, in the case of using the selection reference for the health guidance receivers, can be compared with each other for review, so that the system has an advantageous effect in that the selection reference for the health guidance receivers, capable of gaining a higher health guidance effect, can be made use of. In addition, in the assessment and readjustment routine, the user can refer to the medical care cost in the case of a change in the health guidance parameters, such as a change in the number of the health guidance receivers, and so forth, so that the system has an advantageous effect in that an efficient health guidance plan including the selection reference for the health guidance receivers, and a health nurse system can be formulated.

### BRIEF DESCRIPTION OF THE DRAWINGS

FIG. 1 is a block diagram showing one embodiment of a health service support system according to the invention;

FIG. 2 is a view showing an example of health checkup data units managed by a health checkup data management routine;

FIG. 3 is a view showing an example of health guidance data units on actual results of health guidance provided, managed by a health guidance data management routine;

FIG. 4 is a view showing an example of medical fee receipt data units managed by a medical fee receipt data management routine;

FIG. 5 is a view showing an example of data units on health guidance models, managed by a health guidance model management routine;

FIG. 6 is a view showing an example of personal data units on the insured as individuals, managed by a personal data management routine;

FIG. 7 is a view showing an example of company data units, managed by a group information management routine;

FIG. 8 is a flow chart showing a processing procedure for the health service support system according to the invention, in its entirety;

FIG. 9 is a flow chart showing a detailed processing procedure for the step of creating a health guidance model;

FIG. 10 is a flow chart showing an example of a processing procedure in a health guidance receiver selection step 802 in FIG. 8;

FIG. 11 is a view showing an example of a relationship among respective data units when a health guidance plan is formulated;

FIG. 12 is a view showing an example of a relationship among the respective data units when health guidance is executed based on the health guidance plan;

FIG. 13 is a view showing an example of a relationship among the respective data units when the health guidance plan is readjusted;

FIG. 14 is a view showing an example of a screen to enable a user to select and set checkup items and threshold parameters for assessment of health guidance effects;

FIG. 15 is a view showing an example of an items and parameters selection screen to enable the user to select items and parameters for selection of persons who should receive health guidance;

FIG. 16 is a view showing an example of a screen for use in selecting the health guidance receivers;

FIG. 17 is a sequence diagram showing data exchange between a health service support terminal and a database;

FIG. 18 is a view showing an example of an items and parameters selection screen displaying time series health guidance effect, and the number of persons, on the category-by-category basis;

FIG. 19 is a view showing an example of a detailed flow chart of a step for computing the time series health guidance effect, in FIG. 9;

FIG. 20 is a view showing an example of a screen used in the case of making assessment of the health guidance plan;

FIG. 21 is a view showing an example of a screen used in the case of formulating a new health guidance plan; and

FIG. 22 is a flow chart showing an example of a processing procedure in the case of making readjustment of the health guidance plan.

### DETAILED DESCRIPTION OF THE PREFERRED EMBODIMENTS

Preferred embodiments of the invention are described in detail hereinafter with reference to the accompanying drawings. In the following description, there is described a case where a health insurer executes health service with the use of an embodiment of a health service support system according to the invention on the assumption that the health insurer is an organization practicing health service, and the health insurer provides health guidance to employees of a plurality of companies and business units thereof, as groups belonging to the health insurer, by sending health nurses to the respective business units as part of the health service. It is to be pointed out, however, that the invention is not limited in application to those targets for the health service, and the invention may be applicable to a health nurse and a specific group receiving health guidance.

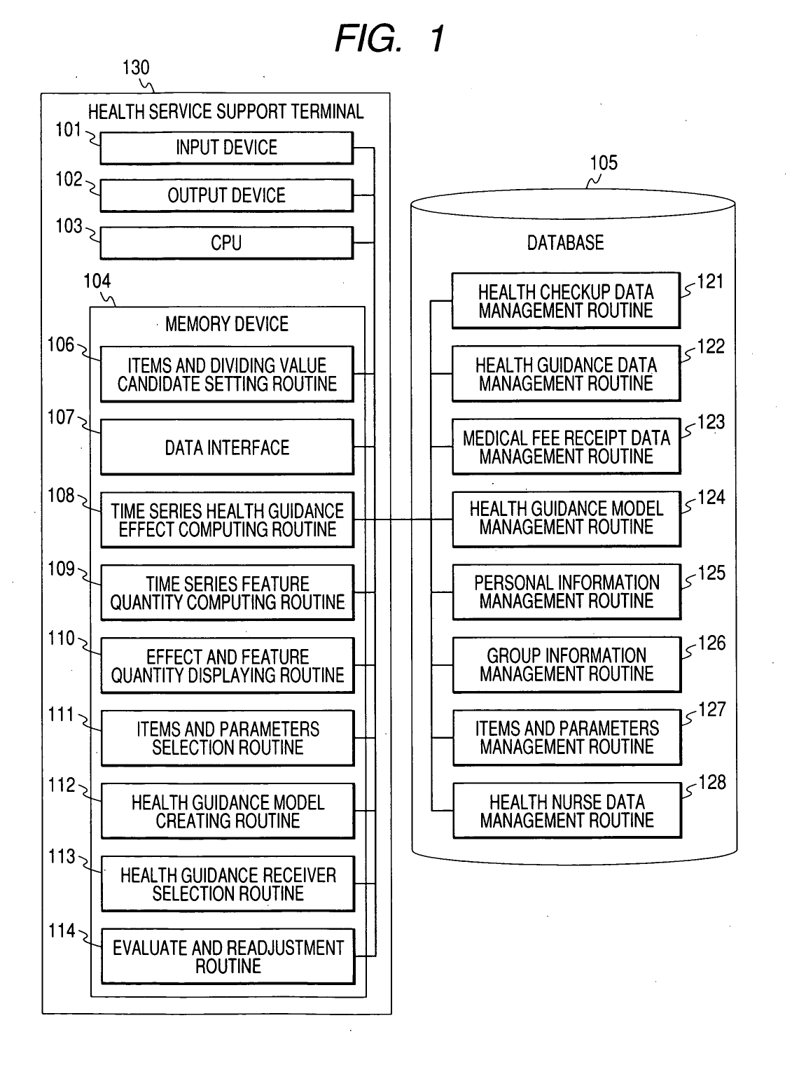

FIG. 1 is a block diagram showing one embodiment of the health service support system according to the invention. The health service support system includes a health service support terminal 130, and a database 105. The health service support terminal 130 is a computer including an input device 101 including a mouse and a keyboard, an output device 102 including a display and a printer, a CPU 103 for operation/execution of programs, and a memory device 104 including a hard disk and a memory. Means such as programs and data, described hereinafter, are stored in the memory device 104 and are read by the CPU 103 as necessary to be thereby executed.

In the memory device 104, there are stored an items and dividing value candidate setting routine 106 for setting health checkup data items as candidate items serving as a selection reference for health guidance receivers, a data interface 107 for getting health checkup data, health guidance data, and medical fee receipt data from the database 105, a time series health guidance effect computing routine 108 for computing health guidance effects on a time-series basis by the category combining items and dividing value candidates with each other on the basis of the health checkup data, health guidance data, and medical fee receipt data, a time series feature quantity computing routine 109 for computing a feature quantity, such as an inclination, and so forth, from time-series health guidance effect, an effect and feature quantity displaying routine 110 for displaying the time-series health guidance effect and the time-series feature quantity, an items and parameters selection routine 111 for selecting items and parameters to be used, on the basis of the health guidance effects and the feature quantities, as displayed, a health guidance model creating routine 112 for creating a health guidance model deciding the order of priority in health guidance in accordance with the items/parameters, as selected, and a health guidance receiver selection routine 113 for selecting health guidance receivers by use of a health guidance model as created.

Further, the database 105 includes a health checkup data management routine 121 for managing health checkup data, a health guidance data management routine 122 for storing record of health guidance provided, and a medical fee receipt data management routine 123 for storing medical fee receipt data. The database 105 further includes a health guidance model management routine 124 for storing the health guidance model displaying the order of priority in health guidance. Still further, the database 105 includes a personal data management routine 125 for recording personal identification numbers, sex, and so forth, a group information management routine 126 for recording addresses of the respective business units where respective persons belong, and so forth, an items and parameters management routine 127, a health nurse data management routine 128 for managing data on persons giving health guidance such as health nurses, and so forth.

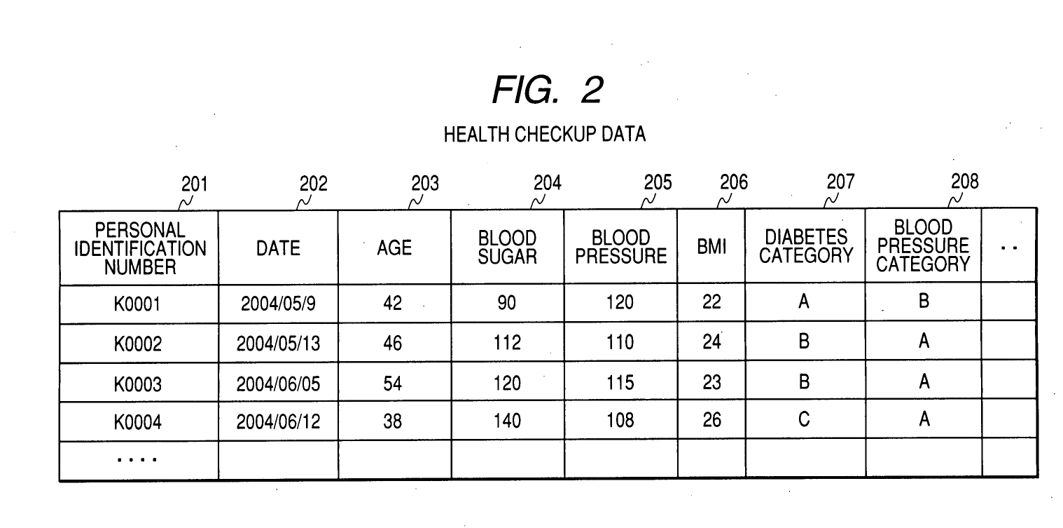

FIG. 2 is a view showing an example of the health checkup data units managed by the health checkup data management routine 121. Management is made on the health checkup data including final test values such as blood sugar 204, blood pressure 205, and BMI 206, results of determination on the basis of test results, made by a physician, such as determination in diabetes category 207, and blood pressure category 208, besides a personal identification number (ID) 201 for identifying individual persons, a health checkup date 202, an age 203 on the health checkup date, and so forth.

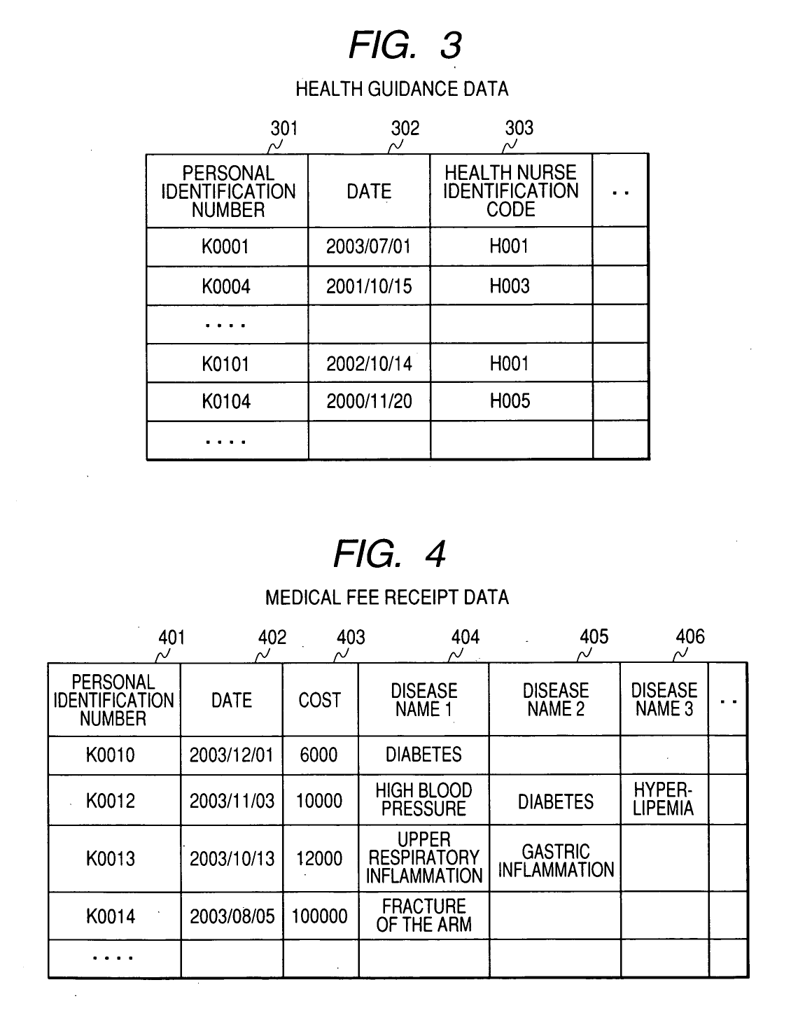

FIG. 3 is a view showing an example of the health guidance data units on actual results of the health guidance provided, managed by the health guidance data management routine 122. Management is made on the health guidance data including a personal identification number (ID) 301 for identifying respective persons who received health guidance, a health guidance date 302 indicating a day when the health guidance was provided, a person in charge 303, indicating a health nurse who executed the health guidance, and so forth.

FIG. 4 is a view showing an example of the medical fee receipt data units managed by the medical fee receipt data management routine 123. The medical fee receipt data is data on a medical fee billing statement issued to the health insurer by a medical institution to charge for medical fees when respective persons receive a medical examination by the medical institution. Management is made on the medical fee receipt data including a personal identification number (ID) 401 for identifying the respective persons, a medical examination date 402, a cost 403 as data on the medical care cost, and data units 404, 405, 406, on respective disease names in connection with which medical examinations were conducted.

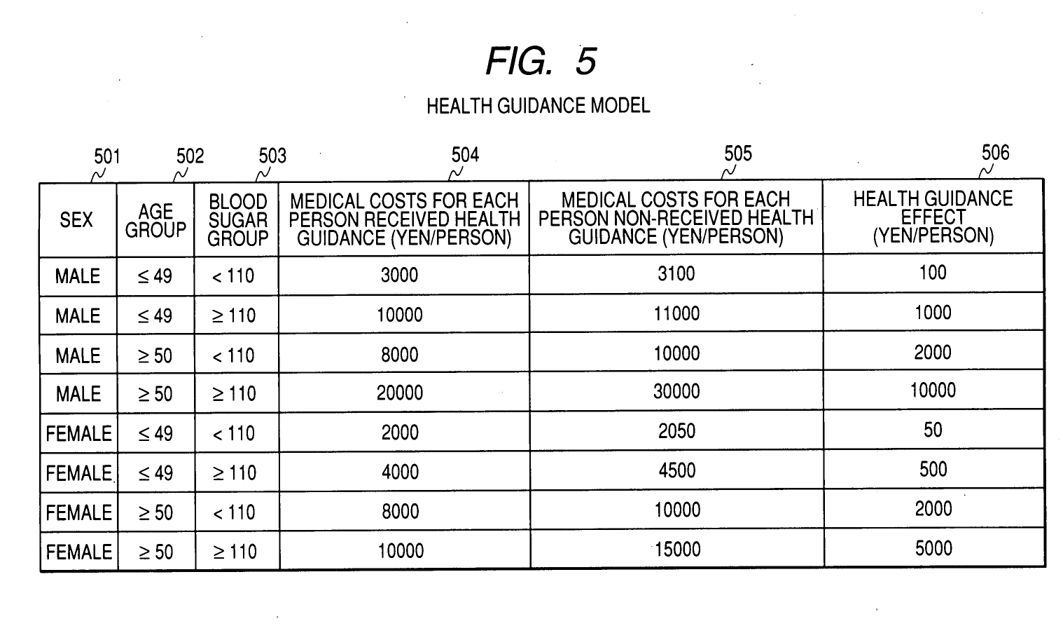

FIG. 5 is a view showing an example of data units on the health guidance models, managed by the health guidance model management routine 124. The health guidance data manages a medical care cost 504 for each person receiving health guidance (yen/person), a medical care cost 505 for each person receiving no health guidance (yen/person), and a health guidance effect 506, against combinations of health checkup items and parameters such as sex 501, age 502, blood sugar 503, and so forth.

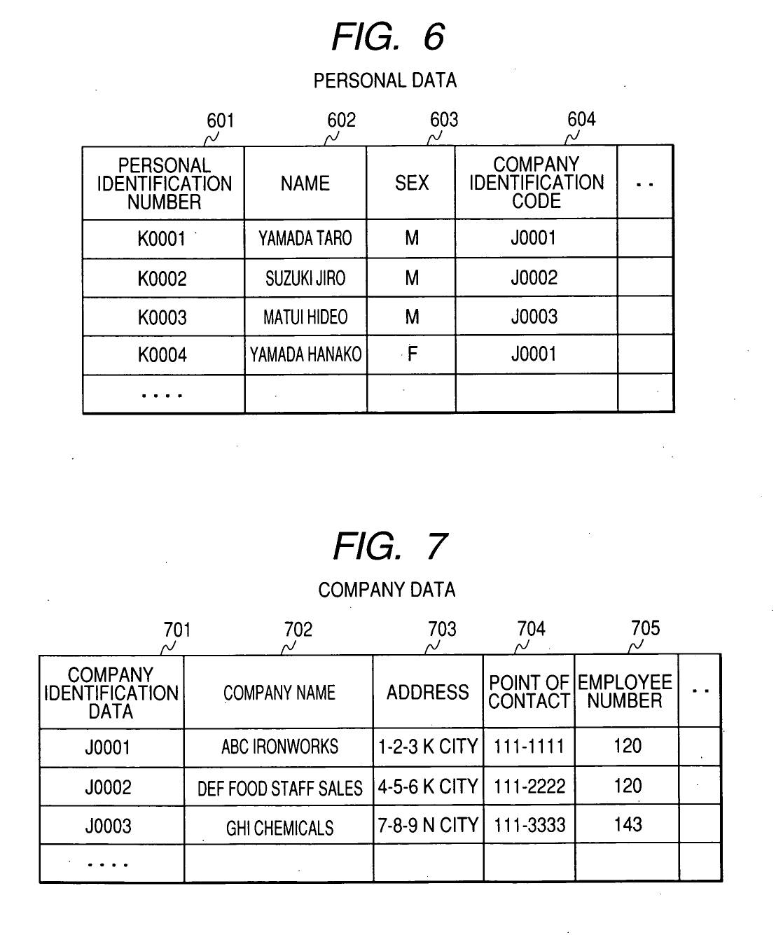

FIG. 6 is a view showing an example of personal data units on the insured as individuals, managed by the personal data management routine 125. Management is made on the personal data including a personal identification number (ID) 601 for identifying individual persons, name 602, sex 603, business unit identification code 604 of business units where the individual persons belong, and so forth.

FIG. 7 is a view showing an example of company data units, managed by the group information management routine 126. Management is made on the company data including a business unit identification code 701, business unit name 702, address 703, point of contact 704 such as telephone number, and so forth, the number 705 of employees of the respective business units, and so forth.

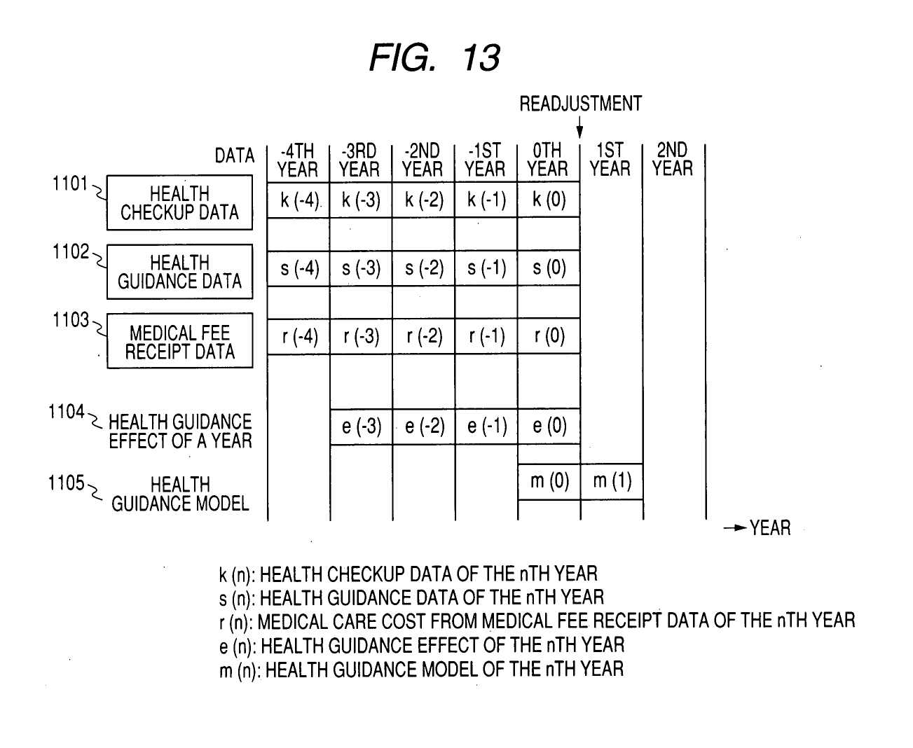

FIGS. 11 to 13 each are a view showing an example of relationships among respective data units when a health guidance plan is first formulated, when health guidance is executed based on the health guidance plan, and when the health guidance plan is readjusted, respectively. In this case, a year serving as a reference is referred to as the 0-th year, a year preceding thereto is referred to as the −1-th year, and a year succeeding thereto is referred to as the 1-th year. Herein, there are shown the relationships among the respective data units in the case where assuming that a year when health guidance is actually executed is the 0-th year, the health guidance plan is developed at the start of the 0-th year (the end of the −1-th year) (refer to FIG. 11), the health guidance based on the plan is executed in the 0-th year (refer to FIG. 12), and actual results are evaluated at the end of the 0-th year, thereby developing a plan for the 1-th year (refer to FIG. 12).

Health checkup data 1101 is health checkup data set in a form shown in FIG. 2, stored in the health checkup data management routine 121 of the database 105. In FIG. 11, it is shown that data units for a period from the −4-th year to the −1-th year are stored, and the health checkup data 1101 of the n-th year is denoted by k (n). Health guidance data 1102 is health guidance data set in a form shown in FIG. 3, stored in the health guidance data management routine 122. Medical fee receipt data 1103 is medical fee receipt data set in a form shown in FIG. 4, stored in the medical fee receipt data management routine 123. As with the health checkup data 1101, it is shown that the health guidance data 1102, and the medical fee receipt data 1103 each represent data units as-accumulated in the period from the −4-th year to the −1-th year, and the health guidance data 1102 of the n-th year is denoted by s (n) while the medical fee receipt data 1103 of the n-th year is denoted by r (n).

Diseases that the health guidance is in an attempt to prevent incidence thereof are lifestyle-related diseases including diabetes, and so forth. As the health guidance prevents persons who have not contracted the lifestyle-related diseases as yet from contracting the diseases, so occurs a difference in incidence between those who have received the health guidance and those who have not received the health guidance, but such a difference will become apparent as beneficial effects in the following year onwards, that is, after some interval in time. In this case, description is given on the assumption that the effect of the health guidance is reflected in the medical care cost of the succeeding year. A health guidance effect of a year (1104) represents a health guidance effect computed on the basis of the health checkup data 1101, the health guidance data 1102, and the medical fee receipt data 1103. In this case, on the assumption that the effect of the health guidance provided on the basis of health checkup is reflected in medical care cost of the succeeding year, the health guidance effect of a year for the n-th year, e (n), is found in view of a medical care cost from the medical fee receipt data for the n-th year, r (n), on the basis of an individual's condition obtained from the health checkup data one year before, k (n−1), and whether or not the health guidance was executed from the health guidance data one year before, s(n−1). A health guidance model 1105 is a model in a form shown in FIG. 5, stored in the health guidance model management routine 124 of the database 105, and is set up on the basis of variation in the health guidance effects of a year, 1104, and so forth. Examples of specific methods of finding the health guidance effects of a year, 1104, and the health guidance model 1105 will be described later.

In FIG. 11, there is shown a state in which the health guidance effects of a year, e (0), is computed from e (−2) at the start of the 0-th year, using the health checkup data, the health guidance data, and the medical fee receipt data, for the period from the −4-th year to the −1-th year, and based on results of such computation, a health guidance model m (0) is set up.

FIG. 12 shows the relationships among the respective data units at a point in time, when the health guidance is actually executed in the 0-th year, and in FIG. 12, there is shown a state in which the persons who should receive the health guidance are selected from the health checkup data of the 0-th year, k (0); in accordance with the health guidance model m (0) to thereby execute the health guidance, which results are being recorded as the health guidance data s (0).

FIG. 13 shows the relationships among the respective data units at a time when the health guidance model m (0) is readjusted on the basis of the results of the 0-th year, after the end of the 0-th year, to thereby create a health guidance model m (1) of the 1-th year.

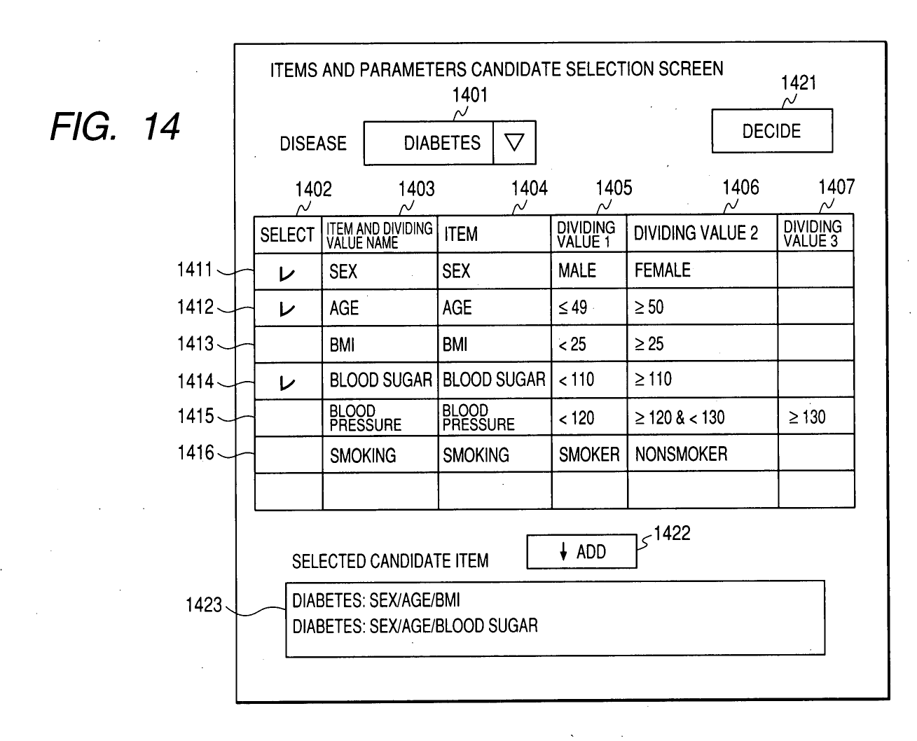

FIG. 14 is a view showing an example of a screen to be displayed on the output device 102 by the items and dividing value candidate setting routine 106 so as to enable a user to select and set checkup items and threshold parameters for assessment of the health guidance effect. A disease input box 1401, and a list of items and parameters are displayed in the screen. The list of the items and parameters displays a select input column 1402, an item and dividing value name column 1403, an item column 1404, and layered dividing values, 1405 to 1407. The content of display represents data items stored in the items and parameters management routine 127 of the database 105, and for example, a candidate item and dividing value 1411 shows that the item of sex is divided into two layers, male and female, in dividing value. Further, an age item and dividing value 1412 shows that age is divided into two groups, one not more than 49, and one not less than 50 in terms of parameters. If an item and dividing value to be combined together are selected in the select input column 1402, and an add-button 1422 is pressed, the item and dividing value is displayed in a list 1423 of selected candidate items.

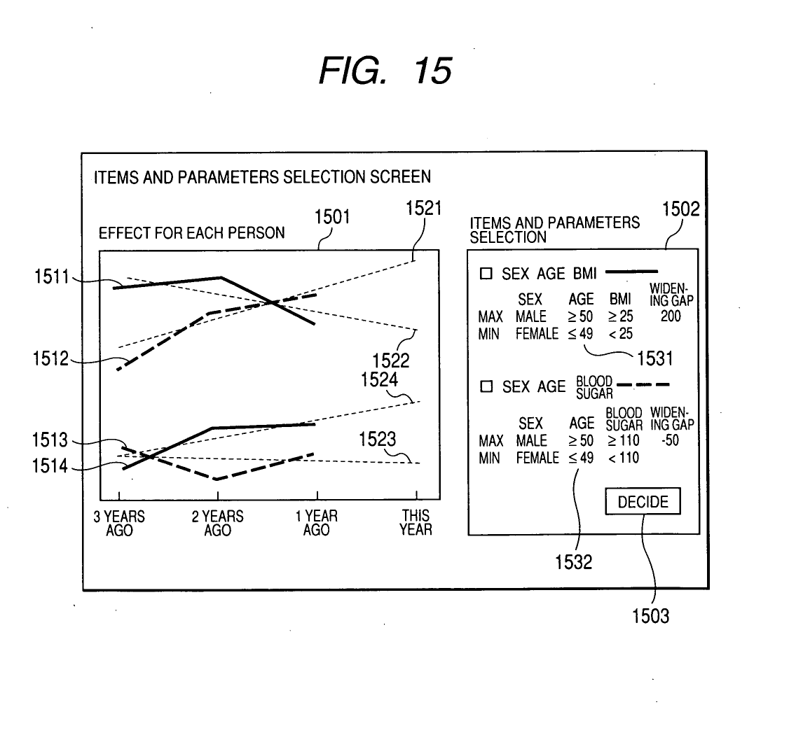

FIG. 15 is a view showing an example of an items and parameters selection screen to be displayed on the output device 102 by the effect and feature quantity displaying routine 110 so as to enable a user to select the items and parameters for selection of the persons who should receive the health guidance. In this case, the time-series health guidance effects for respective persons are displayed (1501), displaying the effects of categorizing items and dividing value in the case of using sex, age, and BMI, and the effects the effects of categorizing the items and dividing value in the case of using sex, age, and blood sugar. In a column 1502 for items and parameters selection, there are displayed the items and parameters that are available, thereby enabling the user to select which of the items and parameters are to be used.

Reference numerals 1511, 1512, 1513, 1514 indicate transition of the health guidance effect of a year, 1104, e (n), as shown in FIG. 11. The reference numerals 1511, 1514 indicate the health guidance effect of a year in the case of using sex, age, and BMI as the item and parameters, and 1511 indicates the effect of a category of a male at an age not less than 50 and BMI not less than 25, 1514 indicating the effect of a category of a female at an age not more than 49 and BMI less than 25. Further, the reference numerals 1512, 1513 indicate the health guidance effect of a year in the case of using sex, age, and blood sugar as the items and parameters, and 1512 indicates the effect of a category of a male at an age not less than 50 and blood sugar not less than 110, 1514 indicating the effect of a category of a female at an age not more than 49 with blood sugar less than 110. A graph displayed herein indicates the effects of the categories whose effects are at the maximum, and the effects of the categories whose effects are at the minimum. In the graph 1501, reference numerals 1521, 1522, 1523, 1524 denotes approximate straight lines of the transition of the health guidance effects of a year, represented by 1511 to 1514, respectively. In the column 1502 for the items and parameters selection, there are displayed the items and parameters, enabling selection to be made out of two categories of the items and parameters, that is, the category 1531 of sex, age, and BMI, and the category 1532 of sex, age, and blood sugar. Selection is made on which of the two groups of the items, and parameters is used, and upon making a decision, a decision-button 1503 is pressed.

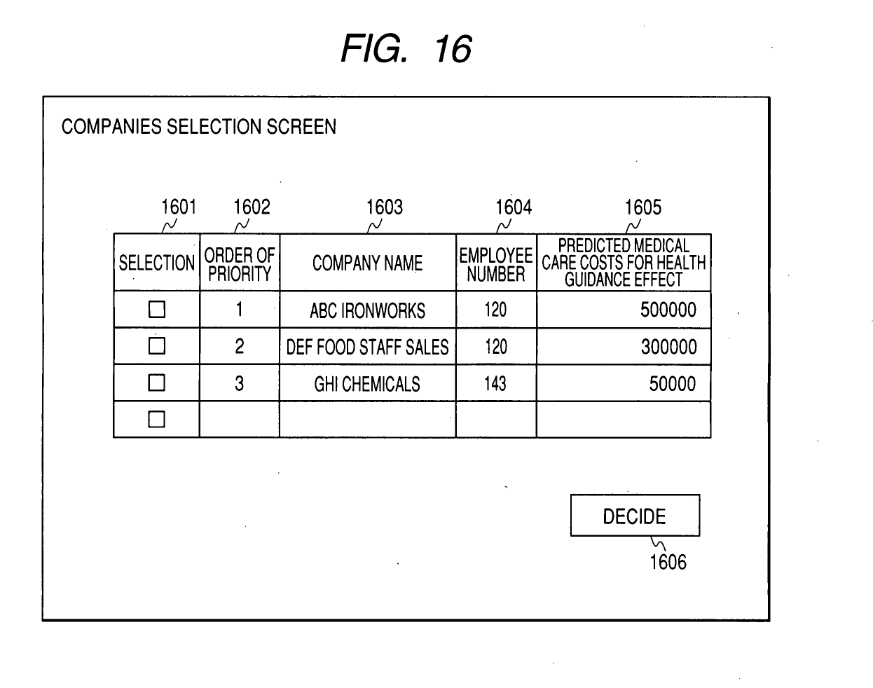

FIG. 16 is a view showing an example of a screen in the case of the health guidance receiver selection routine 113 for selecting the health guidance receivers by displaying the same on the output device 102. On the screen, there are displayed a select input column 1601, the order of priority 1602 in the health guidance, decided on the basis of the health guidance model shown in FIG. 5, business unit name 1603, the number of employees 1604, and guidance effect 1605 in the case of the respective business unit being provided with the health guidance.

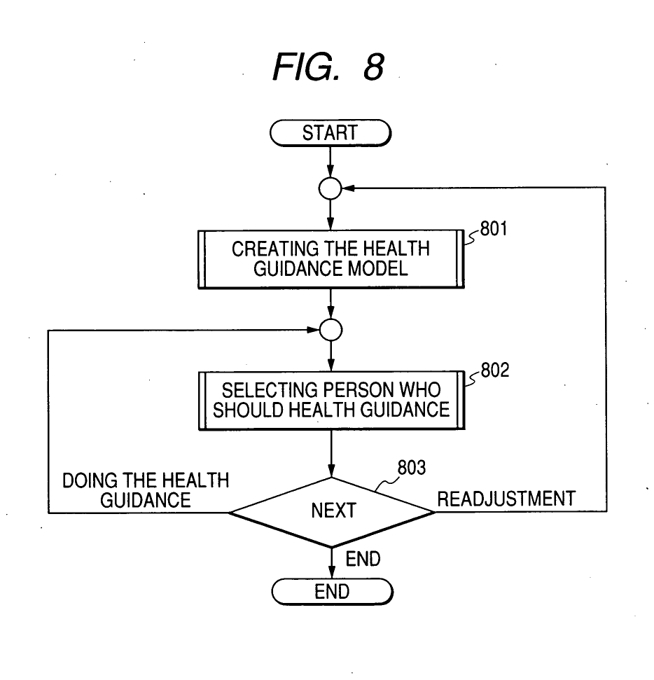

Now, operation is described in detail with reference to flow charts, and a sequence diagram. FIG. 8 is a view showing a flow chart for the health service support system according to the invention, in whole. The system includes a step 801 for creating the health guidance model, and a health guidance receiver selection step 802, and those steps are repeated. For example, the step 801 for creating the health guidance model is taken once a year, and the health guidance receiver selection step 802 is taken once a month.

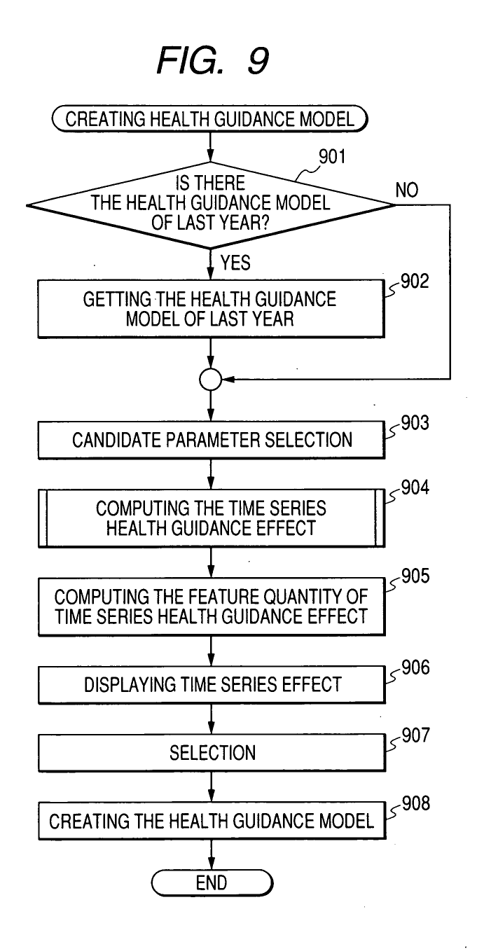

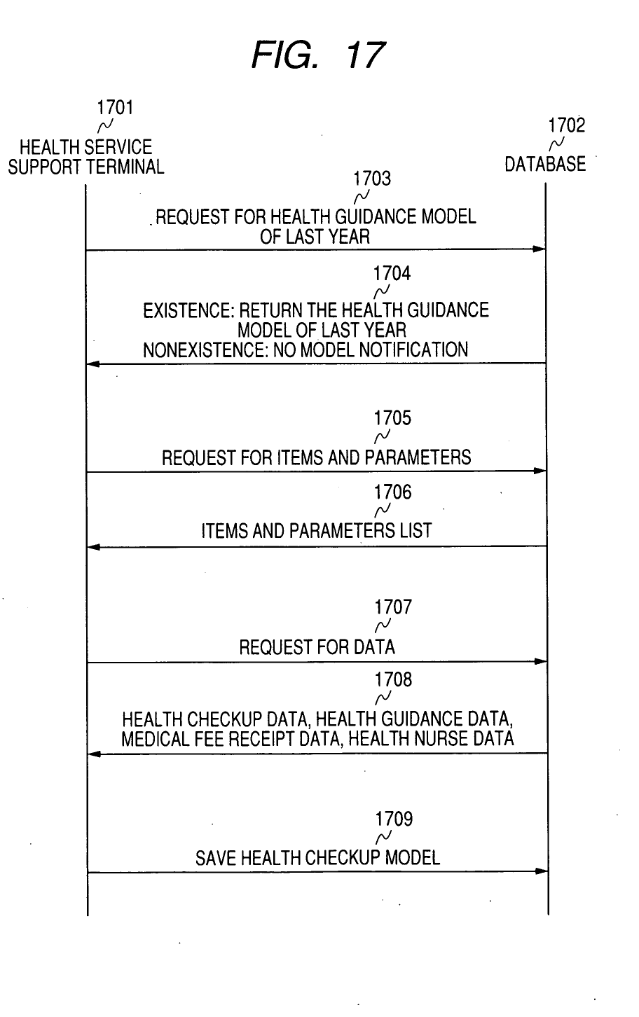

Next, processing in the step 801 for creating the health guidance model is described in detail by way of example, with reference to a flow chart of FIG. 9, a flow chart in FIG. 19, and a sequence diagram in FIG. 17.

First, with the health checkup data, the health guidance data, and the medical fee receipt data, being in a state shown in FIG. 11, a procedure for creating the health guidance model as the health guidance plan of the 0-th year is described hereinafter on the assumption that the 0-th year is the year 2005. FIG. 9 is a view showing an example of a detailed flow chart of the step 801 for creating the health guidance model, in FIG. 8. FIG. 19 is a view showing an example of a detailed flow chart of a step 904 for computing the time series health guidance effect, in the flow chart shown in FIG. 9. Further, FIG. 17 is the sequence diagram showing data exchange between the health service support terminal 130 and the database 105, taking place in the flow chart shown in FIG. 9.

First, in a step 901, checking is made on whether or not the health guidance model for the preceding year exists. If the health guidance plan is developed in the preceding year, the health guidance model of the preceding year is acquired from the health guidance model management routine 124 of the database in a step 902 for acquiring the model. In this case, assuming that the plan is formulated for the first time, the operation proceeds to a step 903. In the sequence diagram in FIG. 17, a request for the health guidance model of the preceding year is made (1703) from a health service support terminal 1701 to a database 1702, and if the health guidance model of the preceding year exists, it is returned while in the case of nonexistence of the health guidance model of the preceding year, notification to the effect of nonexistence of the model is given (1704).

A candidate parameter selection step 903 is a step for the items and dividing value candidate setting routine 106 to display the screen of FIG. 14 to thereby enable the user to input candidate items and dividing value. The items and dividing value candidate setting routine 106 acquires data units on items and parameters from the items and parameters management routine 127 of the database 105, thereby displaying the screen of FIG. 14 on the output device 102. In the items and parameters management routine 127, there are stored final test values included in the health checkup data, such as sex, age, BMI, blood sugar, blood pressure, and so forth, lifestyle-related data such as smoking habits, exercise habits, and so forth, and resultant layered parameters thereof. In the sequence diagram, there is shown a flow of the data exchange such that a request 1705 for the items and parameters list is sent out from the health service support terminal 1701 and the items and parameters list 1706 is sent back thereto. The user operates the input device 101 and selects first a target disease in the disease input box 1401 to thereby select candidate items and parameters. Assuming a case where the user selects diabetes as the target disease on which the user is desirous of getting beneficial effects of the health guidance, the user selects the items and dividing value candidates by the sex (1411), by the age (1412), and by BMI (1413), respectively, in the select input column 1402, and presses the add-button 1422, whereupon the selected candidate items are saved in the selected candidate item list 1423. Further, as other items and dividing value candidates, there are added the items and dividing value candidates by the sex (1411), by the age (1412), and by the blood sugar (1414), respectively. A target disease selected in this case is a disease on which the user is desirous of getting beneficial effects of the health guidance. Upon pressing a decision-button 1421 on the screen, the operation proceeds to the next step.

The next step is the step 904 for computing the time series health guidance effect. An example of processing in the step 904 for computing the time series health guidance effect is described with reference to the flow chart in FIG. 19.

First, in step 1901, the health checkup data, health guidance data, and medical fee receipt data, for a period from the −n-th year to the preceding year, are acquired from the database 105. In this case, on the assumption that the present is the year 2005, the data interface 107 acquires data for the past 4 year (from the year 2001 to 2004) from the health checkup data management routine 121, the health guidance data management routine 122, and the medical fee receipt data management routine 123 of the database 105. In the sequence diagram, there is shown a flow of the data exchange such that the health service support terminal 1701 sends out the request 1705 for the data to the database 1702, whereupon the health checkup data shown in FIG. 2, the health guidance data shown in FIG. 3, and the medical fee receipt data shown in FIG. 4 are sent back to that the health service support terminal 1701 (1708).

Then, the time series health guidance effect computing routine 108 computes the health guidance effect. Assuming in this case that results of the health guidance given on the basis of health checkup results is reflected in medical care cost for the succeeding year, the health guidance effect of the n-th year, e (n), is computed from the health checkup data k (n−1), the health guidance data s(n−1), and the medical fee receipt data r (n). Further, in order to review time series changes, the health guidance effects for 4 years, 3 years, 2 years, and 1 year before, respectively, are computed.

First, in step 1902, it is assumed that i=−4. Then, in step 1903, the health checkup data 4 years before (for the year 2001), k (−4), is categorized by the candidate items and dividing value, using sex, age, and BMI, and by the candidate items and dividing value, using sex, age, and blood sugar. More specifically, data units on the health checkup date 202 in the year 2001, the personal ID 201, the personal identification number 601, the sex 603, the age 203, BMI 206, the blood sugar 204 are fetched from the health checkup data in FIG. 2, and the personal data in FIG. 6. Then, 8 categories formed by various combinations of sex, age, BMI, male/female, age at not more than 49/not less than 50, and BMI less than 25/not less than 25, and another 8 categories formed by similar combinations except for use of sex, age, blood sugar, thereby forming 16 categories.

Subsequently, in step 1904, one of the categories is further categorized according to whether or not the health guidance exists on the basis of the health guidance data s (−4). The personal ID 201 of an individual belonging to the category of a male at the age not more than 49, and BMI less than 25 is extracted from the health guidance data, and the health guidance data on the personal ID 301 for the identical individual, and the health guidance date 302 being in the year 2001 is fetched from the health guidance data in FIG. 3, thereby discriminating persons who received the health guidance as ones having the health guidance from persons who did not receive the health guidance as ones having no health guidance.

Subsequently, in step 1905, a medical care cost data is fetched from the medical fee receipt data r (−3) to thereby sum up medical care costs of the respective categories according to whether or not the health guidance exists. Since it is assumed that the effects of the health guidance are reflected in medical care cost of the succeeding year, there is fetched a medical fee receipt with description stating that the medical examination date 402 according to the medical fee receipt data is in the year 2002, namely, a year succeeding to the year when the health checkup data, and the health guidance data were obtained, and diabetes as a disease name is given in the respective disease name data units 404, 405, 406. Then, respective medical care costs in the medical fee receipts of the persons who received the health guidance and the persons who did not receive the health guidance, having diabetes, are summed up against selected items and divided values. At this point in time, there are also summed up the number of the persons who received the health guidance and the number of the persons who did not receive the health guidance, on a category-by-category basis.

Subsequently, in step 1906, the respective medical care costs as summed up according to whether or not the health guidance exists are divided the number of the relevant individuals to thereby compute a medical care cost per person. Then, in a step 1907, the medical care cost per person of the category of the persons who received the health guidance is subtracted from the medical care cost per person of the category of the persons who did not receive the health guidance to thereby compute the health guidance effect. In this case, a difference in medical care cost between those who received the health guidance, and those who did not receive the health guidance can be regarded as a medical care cost cut-back effect, due to the health guidance provided, and in this case, it is possible to find a health guidance effect against the category of the male at the age not more than 49, and BMI less than 25.

The processing in the steps from 1904 through 1907 is repeated against the remaining 15 categories (step 1908) to thereby obtain the medical care cost cutting-back effect per person, due to the health guidance provided, on the category-by-category basis, as a health guidance effect in every category. This effect represents a health guidance effect of a year, e (−3).

Upon completion of computation of the health guidance effect of a year, against all the categories, “1” is added to “i” (i=i+1) to thereby find a health guidance effect of a year, e (−2), from both a health checkup data k (−3), and a health guidance data, s (−3), for the succeeding year (the year 2002) corresponding to 3 years before, and a medical fee receipt data r (−2) 2 years before (the year 2003). Thus, the processing in the steps from 1903 through 1908 is repeated until “i” turns to “2” (“i”=2) (step 1909), thereby obtaining the time series health guidance effect by the category. The step 904 for computing the time series health guidance effect is thus completed.

Subsequently, in a step 905 for computing the feature quantity of time series health guidance effect, the time series feature quantity computing routine 109 computes the feature quantity of time series health guidance effect on the category-by-category basis. Herein, in the case of using the combination of sex, age, and BMI, and the combination of sex, age, and blood sugar, approximate straight lines representing time-dependent changes in health guidance effect, on a category-by-category basis (for example, a category of a male at an age not more than 49 and BMI less than 25), are found for the respective combinations. The approximate straight line is computed by, for example, the method of least squares, and so forth. Thus, the feature quantity of the health guidance effect for each of the categories can be obtained.

Further, a difference in the feature quantity of time series health guidance effect between the respective categories is fetched. Within the categories using the combination of sex, age, and BMI, a category where the latest health guidance effect of a year, e (−1), is at the maximum, and a category where the latest health guidance effect of a year, e (−1), is at the minimum are found out. For example, if the category with the latest health guidance effect of a year, e (−1), being at the maximum is a category of a male at an age not less than 50 and BMI not less than 25 while the category with the latest health guidance effect of a year, e (−1), being at the minimum is a category of a female at an age less than 49 and BMI less than 25, a widening gap between the respective approximate straight lines of those two categories is fetched. With the respective categories with the health guidance effect of a year, one year before, (of the year 2004) being at the maximum, and at the minimum, the health guidance effect one year before, (of the year 2004), and a health guidance effect of the current year (of the year 2005) are found on the basis of the approximate straight lines representing the health guidance effect. Then, there are found values obtained by subtracting the health guidance effect of the category having the minimum guidance effect from the health guidance effect of the category having the maximum guidance effect, for the preceding year, and the current year, respectively, and a difference in the health guidance effect for the succeeding year is subtracted from a difference in the health guidance effect for the current year to thereby find a widening gap in the effect between the category having the maximum health guidance effect, and the category having the minimum health guidance effect. If a difference between the category having the maximum health guidance effect, and the category having the minimum health guidance effect is wider, it can be considered that the features of the health checkup results, as expressed by the items and parameters, represent parameters that the category capable of gaining a high health guidance effect when the guidance is given can be better distinguished from the category gaining a low health guidance effect when the guidance is given. Further, a widening gap in the effect can be regarded to represent parameters on which a sharper distinction between persons having a high health guidance effect, and those having a low health guidance effect can be made in the future.

Subsequently, in a time series effect displaying step 906, the effect and feature quantity displaying routine 110 displays the health guidance effect, and time series feature quantity, as computed, and a screen shown in FIG. 15 is displayed on the output device 102. In this case, a health guidance effect per person on the category-by-category basis is displayed on the time-series basis (1501), and in the case of using a combination of sex, age, and BMI, there are displayed a combination 1511 having the maximum health guidance effect (for example, a combination of a male at an age not less than 50 and BMI not less than 25), and a combination 1514 with the minimum health guidance effect (for example, a combination of a female at an age less than 49 and BMI less than 25), and the respective approximate straight lines thereof 1522, 1524 are also displayed.

Further, in the case of using a combination of sex, age, and blood sugar, there are displayed a combination 1512 having the maximum health guidance effect (for example, a combination of a male at an age not less than 50 and blood sugar not less than 110), and a combination 1513 having the minimum health guidance effect (for example, a combination of a female at an age less than 49 and blood sugar less than 110), and the respective approximate straight lines thereof 1521, 1523 are also displayed. In the column 1502 for items and parameters selection, there are displayed data units on the contents of the items and parameter, and a widening gap in the health guidance effect with respect to two combinations 1531, 1532, respectively, thereby urging the user to input a decision on which of the combinations is used.

Then, in a selection step 907, the user watches the graph to thereby examine which of the combinations is to be selected so as to be able to extract a category higher in the health guidance effect. Herein, two data units on the contents of the items and parameter are compared with each other, thereby selecting either one thereof.

For example, if the combination 1512 of a male at the age not less than 50 and blood sugar not less than 110 is used in the category 1532 of sex, age, and blood sugar, the effect one year before will be higher than that for the case of the combination 1511 of a male at an age not less than 50 and BMI not less than 25 being used in the category 1531 of sex, age, and BMI (1). Looking at the respective approximate straight lines, the combination 1512 of a male at the age not less than 50 and blood sugar not less than 110 is seen rising over time, indicating a good possibility of a high effect being obtained the current year as well (2). Looking at the widening gap in the health guidance effect, in the case of using the combination of sex, age, and blood sugar, a difference in the effect between the category with the health guidance effect at the maximum, and the category with the health guidance effect at the minimum is found greater, and a gap therebetween is found widening (3), thereby verifying a basis on which a sharper distinction can be made between the category with a health guidance effect, and the category with a low health guidance effect. Hence, the case of using the combination of sex, age, and blood sugar is selected as the basis for selecting the persons who should receive the health guidance, whereupon the category 1532 is selected, and the decision-button 1503 is pressed.

Subsequently, in a health guidance model creating step 908, the health guidance model creating routine 112 creates the health guidance model shown in FIG. 5 on the basis of a combination of the selected items/parameters. Since the health guidance model has selected the combination of sex, age, and blood sugar, the medical care cost 504 for each person receiving health guidance, the medical care cost 505 for each person receiving no health guidance, and the health guidance effect 506 for each person, in the first year, are stored in the respective groups of sex, age, and blood sugar, and the health guidance model thus created is a health guidance model m(0) of the 0-th year, whereupon the health guidance model creating step 908 is completed.

As described in the foregoing, with the health service support system according to the invention, the health guidance effect by the category where the items such as age, sex, test results, lifestyles, and so forth, obtained from the health checkup data, and so forth, are combined with the parameters, and transition of the health guidance effect are found on the basis of the health checkup data, health guidance data, and medical fee receipt data, in the past, by the time series health guidance effect computing routine. By so doing, it is possible to obtain an effect of selecting categorizing parameters for extracting the persons who should receive the health guidance in order to obtain a higher health guidance effect. If the persons who should receive the health guidance are selected particularly by selection of categorizing parameters on which a difference between the health guidance effect at the maximum, and the health guidance effect at the minimum is greater, this will enable a sharper distinction to be made between persons having a high health guidance effect, and persons having a low health guidance effect. Further, if the persons who should receive the health guidance are selected by adoption of categorizing parameters on which the health guidance effect continues to rise with time due to transition of the health guidance effect on the category-by-category basis, this will have an effect of enabling persons who can gain a high health guidance effect to be selected as the persons who should receive the health guidance. Still further, if the persons who should receive the health guidance are selected by adoption of categorizing parameters on which a difference in the health guidance effect between those having a high health guidance effect, and those having a low health guidance effect is widened over time, this will enable a sharper distinction to be made between those having a high health guidance effect, and those having a low health guidance effect, thereby enabling persons who can gain a high health guidance effect to be selected as the persons who should receive the health guidance. Accordingly, the health guidance plan adopting the categorizing parameters selected this way as the condition for selecting the persons who should receive the health guidance is effective in obtaining a higher health guidance effect.

Further, if the health guidance model for the preceding year is found to exist in the step 901, the health guidance model of the preceding year is acquired from the health guidance model management routine 124 in the step 902, and as to the items and parameters used in the health guidance model of the preceding year in addition to the candidate parameters selected in the step 903, there are executed computation of the time series health guidance effect in the step 904, computation of the feature quantity of time series health guidance effect in the step 905, and displaying of time series effects in the step 906, thereby comparing the items and parameters used in the health guidance model of the preceding year with items and parameters newly adopted as a candidate. Hence, the system has an advantageous effect in that it is possible to compare the items and parameters used in the health guidance model of the preceding year with the items and parameters newly adopted as the candidate at the time of readjustment of the health guidance plan, thereby selecting which of those items and parameters are preferable as parameters for selection of the persons who should receive the health guidance.

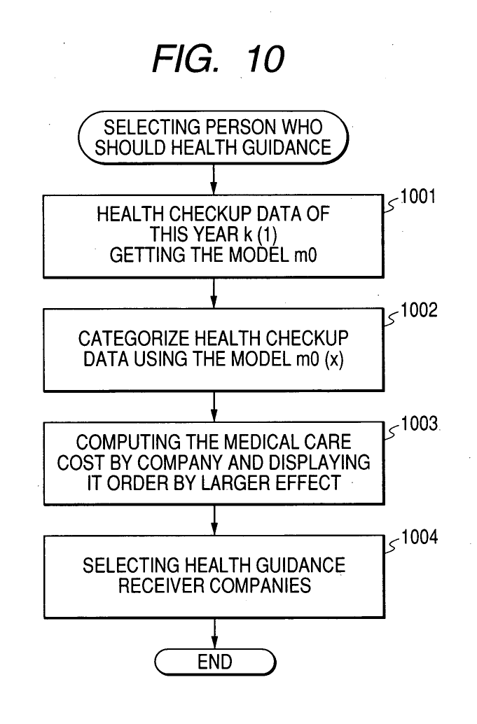

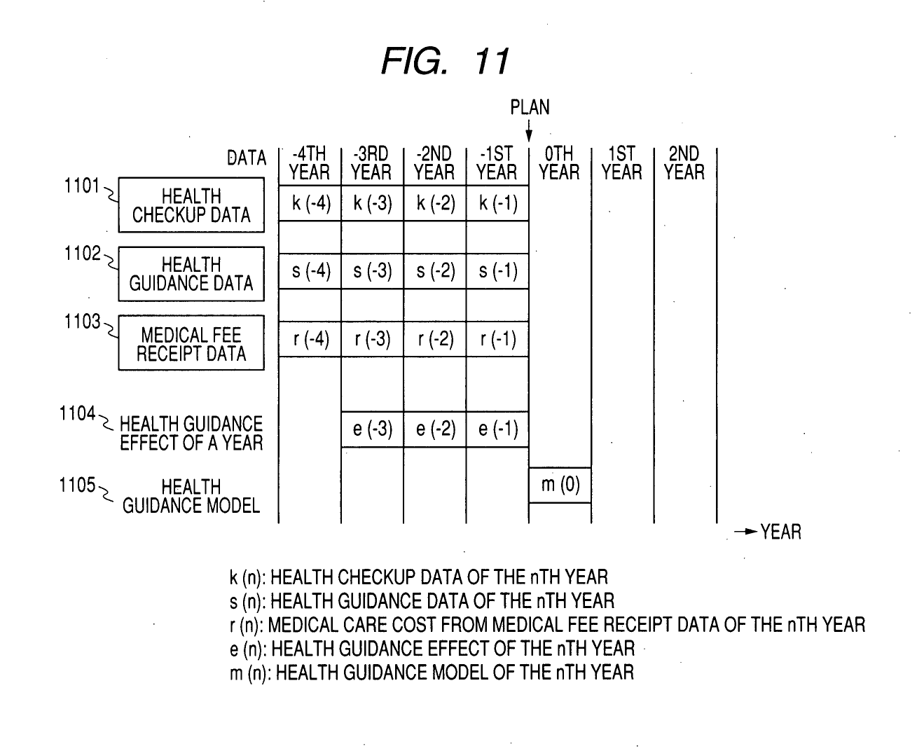

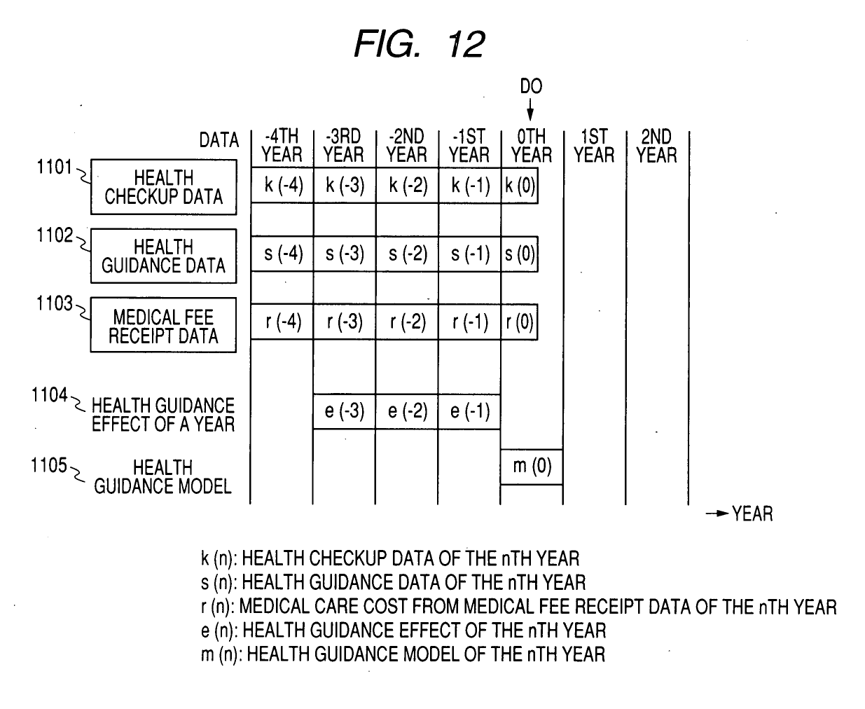

Subsequently, there is described hereinafter specific processing procedure in the health guidance receiver selection step 802 by way of example. FIG. 10 is a flow chart showing an example of the processing procedure in the health guidance receiver selection step 802. The health guidance receiver selection step 802 is a step for specifically deciding who should receive the health guidance during the health guidance executed in a relevant month as previously described. In this case, it is assumed that the health guidance is executed on the basis of a business unit where a person belongs, and a business unit to which the health guidance is given is selected. At a tine when the health guidance is executed, data units are in a state shown in FIG. 12. First, in a step 1001 for acquiring data units, the health guidance receiver selection routine 113 acquires health checkup data k (0) up to the present time point of the current year (the year 23005, the 0-th year) from the health checkup data management routine 121. The health guidance receiver selection routine 113 further acquires data on the health guidance model m (0) as shown in FIG. 5, from the health guidance model management routine 124. The health guidance receiver selection routine 113 still further acquires the personal data shown in FIG. 6 from the personal data management routine 125 and the data on the business units, shown in FIG. 7, from the group information management routine 126, respectively.

Next, in a step 1002, the health guidance receiver selection routine 113 categorizes the health checkup data K (0) using the model m (0). First, on the basis of the personal ID 201 of the health checkup data, the ID 601 of the personal data, the business unit identification code 604 of the personal data, and the business unit identification code 701 of the company data, the health checkup results are divided by the business unit

Further, in the category of the health guidance model m (0), the number of persons is divided. Since the health guidance model m (0) is divided by the sex (male/female), age (not more than 49/not less than 50), and blood sugar (<110/≧110), the number of persons is summed up for every business unit on the category-by-category basis.

Subsequently, in a step 1003, the health guidance receiver selection routine 113 computes a medical care cost and a medical care cost cutback effect for every business unit on the basis of the number of employees of the respective business units by the category. Since the health guidance model m (0) includes the medical care cost 504 for each person in the case of providing a health guidance, and the medical care cost 505 for each person in the case of providing no health guidance, by the sex, age, and blood sugar, a medical care cost for each business unit in the case of providing the health guidance, and the medical care cost 505 for each in the case of providing no health guidance are computed based on the product of the medical care cost for each person and the number of employees for every business unit by the category. A difference in medical care cost between the business unit receiving the health guidance, and the business unit receiving no health guidance is computed by the business unit to be thereby taken as a health guidance effect. Results of such computation are displayed, as a business unit selection screen shown in FIG. 16, on the output device 102. In this case, the business unit names are displayed in a descending order of the health guidance effect, that is, in the order of ABC Ironworks, Def Foodstuff Sales, and GHI Chemicals.

Next, in a step 1004, the user checks the order of priority 1602 and the medical care cost cutback effect 1605 due to the health guidance, as displayed on the screen in FIG. 16, thereby selecting business units to which the health guidance is executed. The user inputs a check mark into respective spots of the select input column 1601, corresponding to selected business units before pressing a decision-button 1606. By the procedure described as above, the business units where the health guidance is actually executed can be decided.

As described in the foregoing, the health service support system according to the invention has an advantageous effect in that if health guidance receiving business units are selected by the health guidance receiver selection routine 113 using the health guidance model as created, this will enable business units capable of obtaining higher health guidance effects to be selected as the health guidance receiving business units. Particularly because the health guidance model in use makes use of a method whereby a sharp distinction can be made between persons having high health guidance effects, and persons having low health guidance effects, it is possible to select business units employing many persons belonging to categories having high health guidance effects. Further, the system has an advantageous effect in that since the health guidance model has data on the medical care cost in the case of receiving the health guidance, and the medical care cost in the case of receiving no health guidance, it is possible to compute a medical care cost cutback effect as anticipated on a business unit basis in the case of receiving the health guidance, so that business units capable of implementing greater cutback in medical care cost can be efficiently selected.

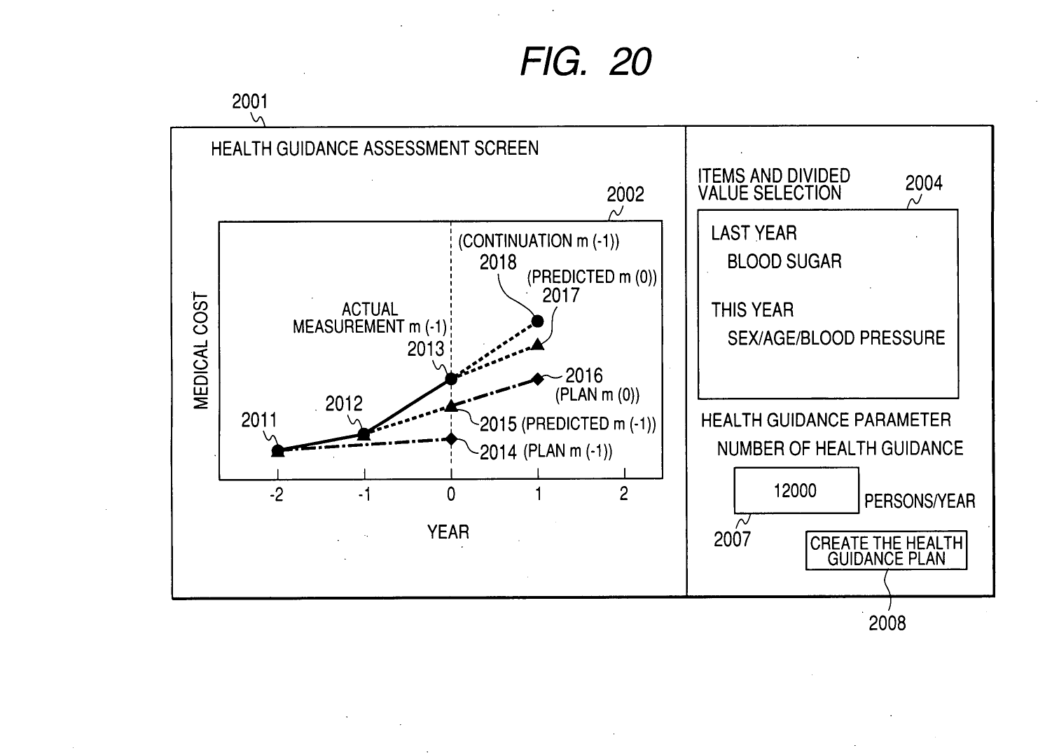

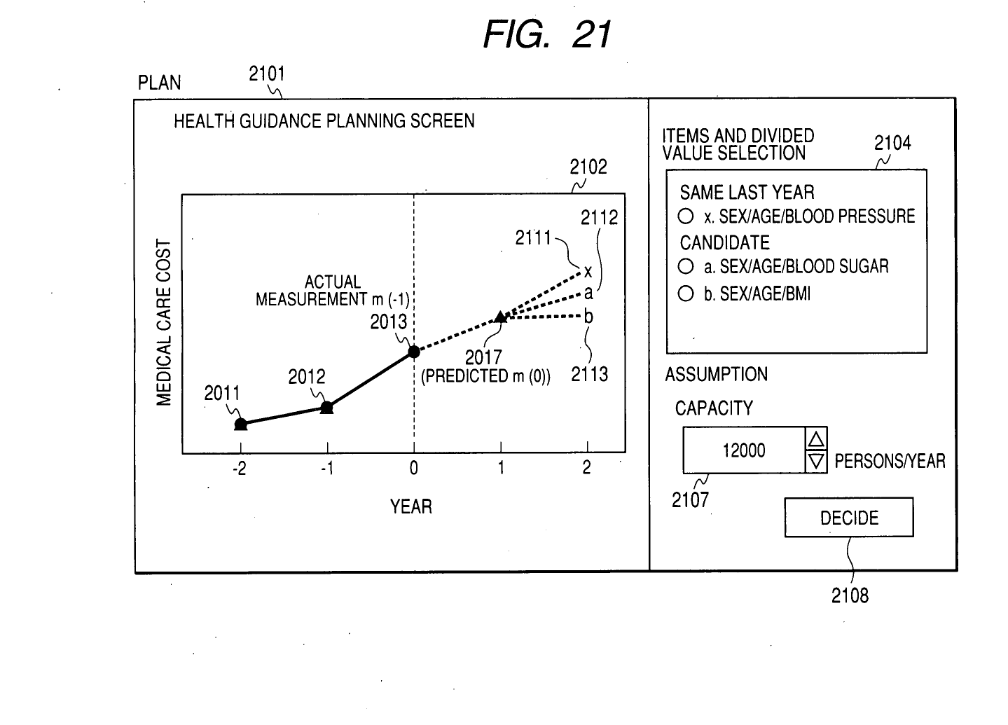

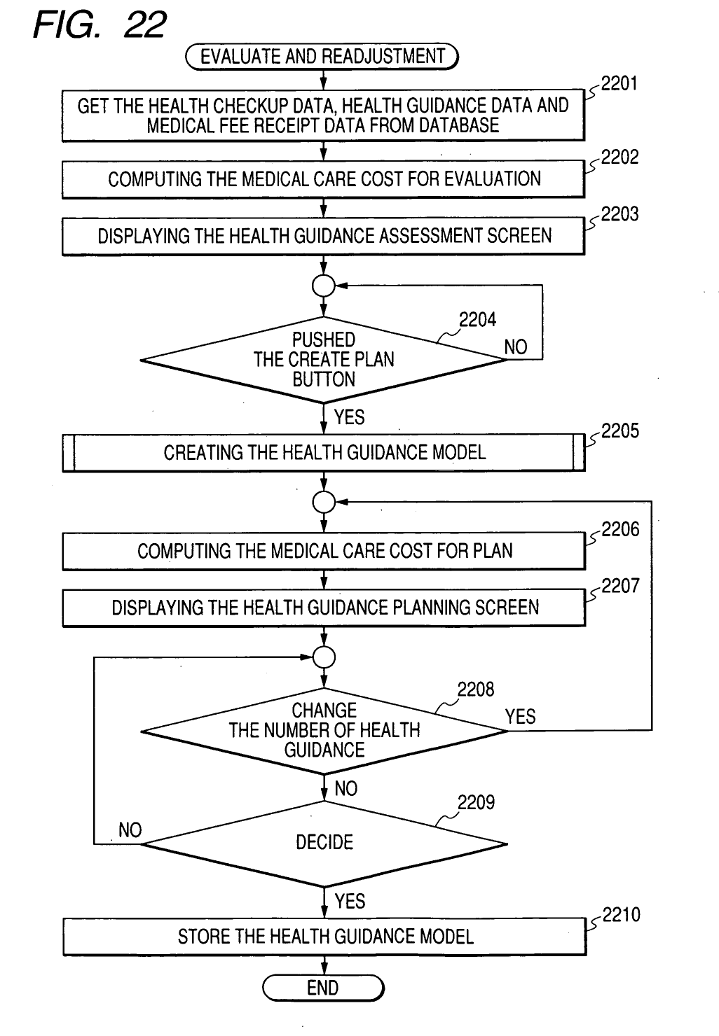

Next, there will be described hereinafter a screen and processing, used in the case where a health service manager of the health insurer makes assessment on health service after execution of the health guidance before making readjustment. In this case, data units are in a state shown in FIG. 13, and it is assumed that a health guidance model m (−1) exists in the (−1)-th year, and in accordance with the health guidance model m (−1), a health guidance data s (−1) is provided. As to the assessment and readjustment, there will be described hereinafter a case where the plans for the (−1)-th year, and the 0-th year, respectively, are readjusted at the end of the 0-th year to thereby formulate a plan for the 1-th year. Description will be given with reference to screens shown in FIGS. 20 and 21, respectively, and a flow chart in FIG. 22.

FIG. 20 is a view showing an example of the screen used in the case of making assessment of the health guidance plan which an assessment and readjustment routine 114 displays on the output device 102. A health guidance plan assessment screen 2001 displays a medical care cost transition graph 2002 of the health insurer in whole, a box 2004 for items and divided value selection for the preceding year and the current year, a box 2007 for the number of the health guidance receivers who are actually guided in the 0-th year, and so forth. There is also displayed a button 2008 for causing transition to a health guidance planning screen.

FIG. 21 is a view showing an example of the screen used in the case of formulating a new health guidance plan which the assessment and readjustment routine 114 displays on the output device 102. The health guidance planning screen 2101 displays medical care cost transition 2102 of the health insurer in whole, an items and divided value selection box 2104, and an a box 2107 for the number of the health guidance receivers. The items and divided value selection box 2104 displays the items and divided value selection for the current year, and items and divided value selection candidates for the succeeding year. The box 2107 for the number of health guidance receivers displays the same number of health guidance receivers that are actually guided in the 0-th year as health guidance parameters, enabling the number of health guidance receivers to be altered. In the items and divided value selection box 2104, either the same items and divided value selection as for the preceding year, or the items and divided value selection candidates can be selected, and upon a decision-button 2108 being pressed, one items and divided value selection as selected in the items and divided value selection box 2104 is decided as the health guidance model m (1) for the 1-th year.

FIG. 22 is a flow chart showing an example of the processing procedure in the case of the assessment and readjustment routine 114 making readjustment of the health guidance plan with the use of the respective screens in FIGS. 20, and 21. In the case of making readjustment of the health guidance plan, in a step 2201 for acquiring data items from the database, the assessment and readjustment routine 114 first acquires the health checkup data managed by the health checkup data management routine 121, the health guidance data managed by the health guidance data management routine 122, the medical fee receipt data managed by the medical fee receipt data management routine 123, and the data items on the health guidance models, managed by the health guidance model management routine 124 via the data interface 107 in order to display the health guidance plan assessment screen.

Next, in a step 2202 for computing the medical care costs for assessment, there are computed transition of the medical care cost, from the past, a forecast medical care cost for the succeeding year, and so forth. The medical care costs computed are of three types, including an actual medical care cost, a forecast medical care cost, and a planned medical care cost. The actual medical care cost is found by summation of the medical care costs of medical fee receipt data for a relevant year. In this case, the actual medical care cost of the 0-th year represents the total of costs according to the medical fee receipt data of the current year, r (0), as in FIG. 13, the actual medical care cost of the (−1)-th year the total of costs according to the medical fee receipt data of the preceding year, r (−1), and the actual medical care cost of the (−2)-th year the total of costs according to the medical fee receipt data for two years before, r (−2). The forecast medical care cost represents a medical care cost one year later as forecast on the basis of the results of the health guidance actually provided. With the present embodiment of the invention, it is assumed that the effects of the health guidance are reflected in the medical care cost for a year succeeding to the year when the health guidance is executed as previously described, so that the medical care cost for the succeeding year is forecast based on the results of the health guidance provided in the current year (the 0-th year). The forecast medical care cost for the 1-th year is computed by categorizing health checkup data for the 0-th year, k (0), according to the items and parameters of the health guidance model m (0), distinguishing the persons who have received the health guidance from the persons who have not received the health guidance on the basis of the health guidance data of the 0-th year, s (0), finding the respective products of the number of the persons in the respective categories, and the respective medical care costs 504, 505 of the health guidance model, and summing up the respective products. The forecast medical care cost for the 0-th year is similarly computed by finding the respective products of the number of the persons in the respective categories, and the respective medical care costs of the health guidance model m (−1) according to which the health guidance is executed in the preceding year.

The planned medical care cost is a medical care cost estimated at a time when the health guidance plan is formulated. The health guidance model m (0) that is used in execution of the health guidance in the current year (the 0-th year) is created at the end of the (−1)-th year, as shown in FIG. 11. Accordingly, on the assumption that the health checkup data of the 0-th year, as forecast at the end of the (−1)-th year, is the same as the health checkup data of the (−1)-th year, and based on the health checkup data of the 0-th year, the health guidance is executed according to the health guidance model m (0), the planned medical care cost for the 1-th year is computed. By finding the product of the number of the health guidance receivers, in the case of selecting the health guidance receivers on the basis of the health checkup data of the (−1)-th year, according to the health guidance model m (0), and the medical care cost 504, and the product of the number of persons other than the health guidance receivers, and the medical care cost 505 according to the health guidance model m (0), the planned medical care cost is found by summing up both the products. The planned medical care cost for the 0-th year is similarly computed. The planned medical care cost is a medical care cost in the case where the effect of the health guidance model is obtained by the persons who have actually received the health guidance. Accordingly, if the health guidance model has a high precision (precision of the effect as obtained), the planned medical care cost will be the same as the actual medical care cost. Further, the planned medical care cost is a medical care cost in the case where the effect of the health guidance model is obtained by the health guidance receivers as assumed at the time of planning. If characteristics of health checkup receivers and the health guidance receivers, assumed at the time of planning, coincide with characteristics of the health guidance receivers in actuality, the planned medical care cost will be the same as the forecast medical care cost.

Next, in a health guidance plan assessment screen display step 2203, there is displayed the health guidance plan assessment screen 2001 shown in FIG. 20. In the medical care cost transition graph 2002, there are displayed the medical care costs computed in the step 2202 for computing the medical care costs for assessment. Herein, there are displayed a medical care cost 2014 estimated at the time of planning the health guidance model m (−1) as the medical care cost of the current year (the 0-th year), and a forecast medical care cost 2015 according to the health guidance model m (−1) forecast at the end of the (−1)-th year, together with the actual medical care costs 2011, 2012, 2013 for the (−2)-th year to the 0-th year (the current year), respectively. Further, there are displayed a medical care cost 2016 estimated at the time of planning the health guidance model m (0) as the panned medical care cost of the succeeding year (the 1-th year), a forecast medical care cost 2017 according to the health guidance model m (0), based on actual guidance results for the current year, and a forecast medical care cost 2018 in the case of selecting the health guidance of the current year according to the health guidance model m (−1) of the preceding year. Further, in the box 2004 for the items and divided value selection, there are displayed the items and divided value selection “blood sugar” adopted in the health guidance model m (−1) of the preceding year, and the items and divided value selection “sex, age, blood pressure” adopted in the health guidance model m (0) of the current year. Further, on the basis of data obtained from the health guidance data, “12000 persons/year” is displayed in the box 2007 for the number of the health guidance receivers.

Having looking at the health guidance plan assessment screen, a person of an health insurer, in charge of making assessment of the health guidance plan, can see transition of the medical care costs to the health insurer, discrepancies among the planned medical care cost, the forecast medical care cost, and the actual medical care cost, for the health guidance provided in the preceding year, and a discrepancy between the planned medical care cost, and the forecast medical care cost, for the health guidance in the current year, to thereby make assessment of the health guidance. For example, the discrepancy between the planned medical care cost, and the forecast medical care cost is associated with a difference in characteristics between the health guidance receivers at the time of planning, and the health guidance receivers in actuality. The person in charge is able to become aware of a possibility that the health guidance receivers cannot be selected as planned when selection is made, or a possibility that distribution in the number of the health guidance receivers, among various categories, varies from that at the time of planning. Further, a discrepancy between the forecast medical care cost and the actual planned medical care cost occurs in the case where there is a difference between an anticipated health guidance effect and an actual health guidance effect. Hence, the person in charge is able to review whether or not the health guidance plan, such as the content of the health guidance, the selection reference, and so forth, should be readjusted.

In the case of the readjustment of the health guidance plan, the user presses the button 2008 for creating the health guidance plan. When the button 2008 for creating the health guidance plan is pressed (a step 2204), a health guidance model creating step 2205 is executed. In the health guidance model creating step 2205, a health guidance model having a high health guidance effect is selected according to the flow chart shown in FIG. 9. In this case, it is assumed that two items and divided value selections including (a) “sex, age, blood sugar”, and (b) “sex, age, blood pressure” are selected for a health guidance model candidate, respectively, and two health guidance models as shown in FIG. 5 are obtained.

Next, in a planed medical care cost computation step 2206, the medical care costs at the time of planning are computed. In this case, computation is made of the medical care costs on the two items and divided value selections taken as the candidate, in the health guidance model creating step 2205. Summation is made of respective medical care costs in the cases of “sex, age, blood sugar” is taken as the items and divided value selection, and “sex, age, blood pressure” is taken as the items and divided value selection, using the health checkup data of the current year (the 0-th year), while 12000 persons as specified in the box 2007 for the number of the health guidance receivers are guided to thereby find the total of the respective medical care costs. Further, summation is made of respective medical care costs similarly selected according to the health guidance model m (0).

Next, in a health guidance planning screen display step 2207, there is displayed the health guidance planning screen 2101 shown in FIG. 21. In the health guidance planning screen, there are displayed the actual medical care costs 2011, 2012, 2013 as computed in the step 2202 for computing the medical care costs for assessment, the forecast medical care cost 2017 according to the health guidance using the health guidance model m (0) of the current year, a medical care cost 2111 computed in the planed medical care cost computation step 2206 in the case of using a health guidance model identical to the health guidance model m (0) of the current year, a medical care cost 2112 in the case of using (a) “sex, age, blood sugar” for the health guidance model candidate, and a medical care cost 2113 in the case of using (b) “sex, age, BMI” for the health guidance model candidate. Further, in the items and divided value selection box 2104, there are displayed the items and divided value selection for the current year, and the two items and divided value selection candidates. Furthermore, as the health guidance parameters, “12000 persons/year” is displayed in the box 2107 for the number of the health guidance receivers.

Subsequently, to take a look at medical care costs in the case of the health guidance parameters being changed, the user inputs so as to alter a numerical value in the box 2107 for the number of the health guidance receivers.

When the numerical value in the box 2107 for the number of the health guidance receivers is altered (a step 2208), the processing reverts to the planed medical care cost computation step 2206, and computes a medical care cost in the case of an increase, or a decrease in the number of the health guidance receivers, whereupon the medical care cost as obtained is displayed in the health guidance planning screen display step 2207.

Then, the user selects the items and divided value selection with which a maximum cutback in medical care cost can be achieved based on the graph in the box 2104 for the items and divided value selection, and subsequently presses the decision-button 2108, whereupon the processing proceeds from the step 2209 to a health guidance model storing step 2210, thereby storing the items and divided value selection as selected in a form shown in FIG. 5 as applicable to the health guidance model for the succeeding year before completion.

As described in the foregoing, with the health service support system according to the invention, the assessment and readjustment routine displays comparison among the planned medical care cost estimated at the time of planning, the forecast medical care cost as forecast after the health guidance is provided, and the actual medical care cost as found one year later, so that the user can make assessment of adequacy of the health guidance model used in the health guidance, adequacy of parameters adopted at the time of creating the model, and adequacy of the content of the health guidance. For example, if the user finds a discrepancy between the planned medical care cost and the forecast medical care cost, this will enable the user to become aware of a difference in tendency existing between the health guidance receivers at the time of planning and those at the time of executing the health guidance. Further, if the user finds a discrepancy between the forecast medical care cost and the actual medical care cost, this will enable the user to become aware of a possibility that a health guidance effect assumed at the time of planning has not been obtained. Because the user can check whether or not the health guidance plan is adequate by checking discrepancies among those medical care costs, the system has an advantageous effect in that the health guidance plan can be readjusted to thereby alter the items and divided value selection for the health guidance receivers to one capable of obtaining a higher health guidance effect.

Further, with the health service support system according to the invention, in the step for creating the health guidance model, health guidance receiver selection parameters under which a sharper distinction can be made between the persons having a high health guidance effect, and the persons having a low health guidance effect can be obtained on the basis of time series transition of the health guidance effect, and further, in the assessment and readjustment routine, the health guidance effects for the health insurer in whole, in the case of using the selection reference for the health guidance receivers, can be compared with each other for review, so that the system has an advantageous effect in that the selection reference for the health guidance receivers, capable of gaining a higher health guidance effect, can be made use of. In addition, in the assessment and readjustment routine, the user can refer to the medical care cost in the case of the health guidance parameters, such as the number of the health guidance receivers, and so forth, being altered, so that the system has an advantageous effect in that an efficient health guidance plan including the selection reference for the health guidance receivers, and a health nurse system can be formulated.

Further, with the embodiment of the invention, described as above, there has been described a case where the number of the health guidance receivers is adopted as the health guidance parameters for use in the assessment and readjustment routine 114, however, the number of the health nurses, the number of the health guidance receivers per health nurse, and so forth may be adopted as the health guidance parameters. Those data units are stored in the health nurse data management routine 128, and are acquired by the assessment and readjustment routine 114 for use. A health guidance plan can be developed so as to be able to gain a higher health guidance effect by altering various parameters concerning the health guidance. Further, the health nurse data management routine 128 is caused to manage cost data on the health guidance to thereby enable medical care cost cutback effects gained to be compared with costs. By so doing, it is possible to develop a health guidance plan whereby the health guidance is executed within confines where the medical care cost cutback effects are gained, and an expenditure of the insurer as a whole can be reduced.

With the embodiments of the invention, described hereinbefore, in the time series health guidance effect computing routine, the health guidance effect per person is found by the category on the time-series basis, and based on the result thereof, there are selected the items and dividing value for selection of the health guidance receivers, however, along with time-series transition of the health guidance effect by the category, transition in the number of persons by the category may be also found to be used for the selection.

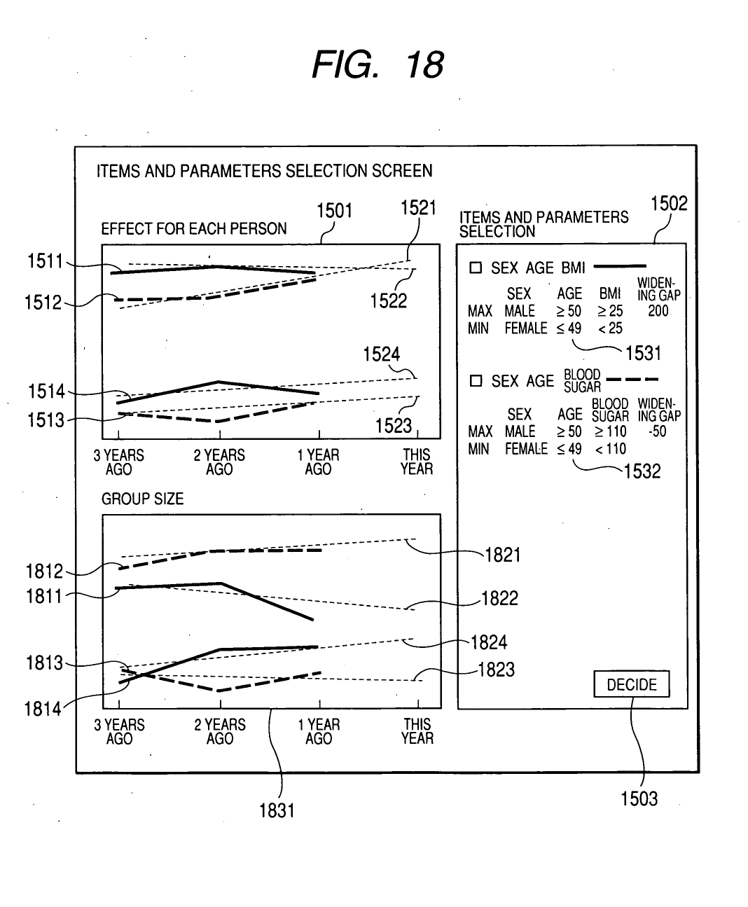

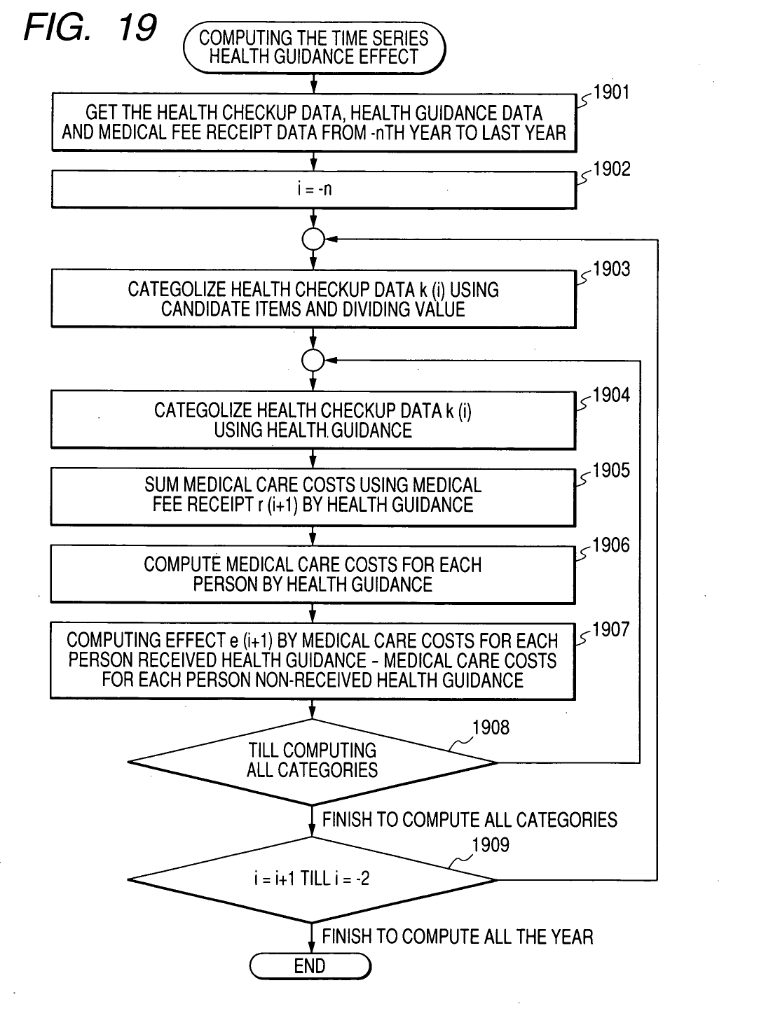

FIG. 18 is a view showing an example of an items and parameters selection screen displaying time series health guidance effect, and the number of persons, on the category-by-category basis. In the figure, there are displayed a graph 1501 for time series health guidance effects per person, and a graph 1831 for group sizes indicating the number of persons belonging to respective categories of identical items and parameters, on a time-series basis. It is shown that in the case of the items and parameters being based on sex, age, BMI, the effect per person in the category of a male at an age not less than 50 and BMI not less than 25 is represented by 1511, and the group size undergoes transition as indicted by 1811 while in the case of the items and parameters being based on sex, age, blood sugar, the effect per person in the category of a male at an age not less than 50 and blood sugar not less than 110 is indicted by 1512 and the group size undergoes transition as indicted by 1812. It is evident that there is not much difference in, for example, health guidance effect per person regardless of the items and parameters, however, as to the group size, there occurs an increase in the group size of the category of a male at an age not less than 50 and blood sugar not less than 110 while there occurs a decrease in the group size of the category of a male at an age not less than 50 and BMI not less than 25. Accordingly, in the case of a group of persons, having a maximum health guidance effect, being provided with the health guidance, the greater the group size, the higher the health guidance effect of the group will be on the assumption that the health guidance effects of the respective persons are substantially equal to each other, so that the items and parameters of sex, age, blood sugar are selected as the items and divided value selection for the health guidance receivers. Thus, the system has an advantageous effect in that not only the transition of the health guidance effects by the category but also the number of the persons by the category can be displayed for comparison with each other, thereby enabling user to decide selection parameters for the health guidance receivers, resulting in higher health guidance.

With the embodiment of the invention, described as above, the effects per person and the group sizes are displayed side by side. However, the product of the effect per person by the group and the group size may be found instead. For example, with respect to data items shown in the screen of FIG. 18, the product of the effect per person indicated by 1511, and the group size indicted by 1811, the product of the effect per person indicated by 1514, and the group size indicted by 1814, the product of the effect per person indicated by 1512, and the group size indicted by 1812, and the product of the effect per person indicated by 1513, and the group size indicted by 1813 may be found. As with the case of the health guidance effect per person, the parameter under which a difference in the effect between the category having the maximum health guidance effect, and the category having the minimum health guidance effect has a greater widening gap is adopted as the selection parameters for the health guidance receivers based on the transition of the health guidance effects of the category in whole and feature quantities thereof. With adoption of this method, it becomes possible to deal with a relationship between the health guidance effect per person, and the group size by the category as a single parameter, so that the items and parameters capable of gaining a higher health guidance can be decided as the selection parameters for the health guidance receivers.

Further, with the embodiment of the invention, described as above, there is described a case, by way of example, where the approximate straight lines representing the respective feature quantities are found by the time series feature quantity computing routine however, besides the approximate straight lines, use may be made of values such as variance representing variation from the approximate straight lines, standard deviation, ad so forth. Since it is anticipated from large variation in the health guidance effect that variation also occurs to the effect obtained if the items and parameters are adopted as the selection parameters for the health guidance receivers, stable health guidance can be expected of the guidance for the current year by adoption of the items and parameters small in variation. Further, besides the approximate straight lines, use may be made of approximate straight lines of high-order function in the time series feature quantity computing routine. Use may be made of methods such as, for example, an exponential approximation method whereby the category rapidly rising in health guidance is approximated, or the exponential approximation method whereby the category slowly rising in health guidance is approximated. By so doing, nonlinear time-series transition of the health guidance effects can be more adequately represented, so that the system can have an advantageous effect in that the items and parameters capable of gaining a still higher health guidance owing to a spread between approximate curves thus obtained can be decided as the selection parameters for the health guidance receivers.

Further, with the embodiment of the invention, described as above, the health guidance receiver selection routine 113 displays the medical care cost cutback effect 1605 for every business unit provided with the health guidance to compare the business units with each other, however, comparison may be made on the basis of a medical care cost cutback effect per person, obtained by dividing the medical care cost cutback effect 1605 by the number of employees 1604. Then, the system has an advantageous effect in that by selecting a business unit having the highest medical care cost cutback effect per person, a business unit capable of efficiently obtaining a high effect can be selected as a target for guidance.

Still further, with the embodiment of the invention, described as above, the user is enabled to make selection with the use of a candidate item selection screen of FIG. 14, displayed by the items and dividing value candidate setting routine 106, and the items and parameters selection screen of FIG. 15, or FIG. 18, displayed by the effect and feature quantity displaying routine 110, however, automatic selection of the items and parameters capable of gaining the highest effect may be adopted instead. A case of automatically creating the health guidance model is described by way of example with reference to the flow chart of FIG. 9. As this is a case of automatic creation, there is adopted a processing procedure wherein the time series effect displaying step 906, shown in FIG. 9, is excluded. If the health guidance model for the preceding year exists in the steps 901, 902, a procedure for acquiring the health guidance model is the same as that shown in FIG. 9.

Next, in the candidate parameter selection step 903, the items and dividing value candidate setting routine 106 acquires data units on the items and parameters from the items and parameters management routine 127 to thereby create a list of candidate parameters. A list of health checkup items, and the layered parameters of values, as indicated by 1411 to 1416 in FIG. 14, is stored in the items and parameters, and the list is acquired. Then, a candidate item list of respective item parameters comprehensively combined together is created. For example, a list completely covering the candidate items such as sex only item 1411, age only item 1412, and combinations of a plurality of items such as sex, age, BMI, blood sugar, and so forth, and all those are adopted as candidate items and parameters. It is set such that limitation is put on the number of combined items, and both persons having received guidance and persons having received no guidance exist in categories formed by combining the items so as to be able to find the health guidance effect. Limitation may be set beforehand to the number of the items, for example, to five items.

Next, in the step 904 for computing the time series health guidance effect, the health guidance effects against the respective candidate items and parameters are computed on a time-series basis by the time series health guidance effect computing routine 108.

Then, in the step 905 for computing the feature quantity of time series health guidance effect, the feature quantities, such as the approximate straight lines, for the health guidance effects against the respective candidate items and parameters are computed on the category-by-category basis by the time series feature quantity computing routine 109.

Next, in the selection step 907, items and parameters where a widening gap between the approximate straight lines corresponding to the respective categories is the largest is selected among a plurality of the items and parameters by the items and parameters selection routine 111.

Subsequently, in the step 908, the health guidance model along with respective values of medical care cost in the case of receiving the health, the medical care cost in the case of receiving no health guidance, the health guidance effect, and so forth, based on the selected items and parameters selected by the health guidance model creating routine 112 are stored in the health guidance model management routine 124, thereby completing the processing of the automatic creation.

As shown in the foregoing, the system has an advantageous effect in that the candidate items and parameters are comprehensively created by the items and dividing value candidate setting routine 106, and the items and parameters where the widening gap between the approximate straight lines representing the guidance effects of the respective categories is the largest is selected among the candidate items and parameters, thereby creating a health guidance model serving as a selection reference for the health guidance receivers, wherein a sharper distinction between the persons having a high health guidance effect, and those having a low health guidance effect can be made, and optimum health guidance effect can be gained by selecting the health guidance receiver from among the categories having a high health guidance effect.

Further, with the embodiment of the invention, described as above, one health guidance model is automatically created, however, if the screen shown in FIG. 21 is used by the assessment and readjustment routine 114, a plurality of health guidance models may be automatically outputted as candidate models. In such a case, several health guidance models may be selected through combinations of several items and parameters where there exists a large gap between the approximate straight lines representing the guidance effects, in descending order of the gap, where the health guidance effect of the category having the maximum guidance effect is high, where the number of persons of categories having a high health guidance, in combination with the group size, is large, and so forth. By so doing, the health guidance models each serving as a selection reference for the health guidance receivers, capable of efficiently gaining a high health guidance, can be obtained from the plurality of the health guidance models as the candidate models.

Still further, the system has an advantageous effect in that if the health guidance model for the preceding year exists, the health guidance plan can be readjusted such that a higher guidance effect can be obtained by selecting items and parameters capable gaining a guidance effect higher than that for the preceding year.

With the embodiments of the invention, described hereinbefore, diabetes is described as an example of diseases for which health guidance is to be provided however, the invention is applicable to other diseases provide that those are lifestyle-related diseases. In the case of the lifestyle-related diseases, change in lifestyle due to health guidance and so forth will lead to prevention of incidence thereof. The lifestyle-related diseases include high blood pressure, hyperlipemia, adiposis, hyperuricemia, and so forth, besides diabetes. Furthermore, the lifestyle-related diseases are said to be associated with cardiopathy, and cerebrovascular disease in the future, and if data over a long term is available, the system can be applicable thereto.

Further, with the embodiments of the invention, described hereinbefore, it has been described that the effect of the health guidance is reflected in the medical care cost of the succeeding year, however, the effect of the health guidance may appear at other periods of time. Since there is case where it takes longer time before the effect of the health guidance appears in the case of lifestyle-related diseases, various periods of time, for example, 3, 5 years later, may be set to check the effect, in which case, the effect of the health guidance can be definitely confirmed. In addition, if a longer period of time is set to check the effect of the health guidance, this will have an advantageous effect in that a health guidance plan can be set up on the basis of a forecast medical care cost over a long term.

Still further, with the embodiments of the invention, described hereinbefore, there has been described a case by way of example where health guidance in health service is executed by the health nurses and so forth, however, use may be made of various health programs besides the health guidance, contributing to prevention of diseases, and promotion of health. The system is applicable to various health services including, for example, service whereby health data is managed via a network to send out advices, nonsmoking program, meal introduction, introduction of sports clubs, and so forth. By so doing, the effects of the various health services on medical care costs can be assessed, and an optimum plan for health service can be formulated to be readjusted as necessary, thereby managing the health service.

What is claimed is:

1. A health service support system comprising:
a database including:
a health checkup data management routine for storing health checkup data units of persons;
a health guidance data management routine for storing health guidance data units of the persons; and
a medical fee receipt data management routine for storing medical fee receipt data units including data units on medical care costs of the respective persons;
a routine for setting not less than two items and dividing value candidates combining health checkup data items for creation of a health guidance model with at least not less than two items and parameters having parameters for layering of the health checkup data items;
a data interface for getting the health checkup data units of the persons, the health guidance data units of the persons, and the medical fee receipt data units, corresponding the items and dividing value candidates, respectively, from the health checkup data management routine, the health guidance data management routine, and the medical fee receipt data management routine, respectively;
a routine for computing a health guidance effect on a time-series basis by the category obtained by combining the items and dividing value candidate with the items and dividing value candidate on the basis of the health checkup data units of the persons, the health guidance data units of the persons, and the medical fee receipt data units;
a routine for computing a feature quantity, in the form of an approximate straight line, from the time-series health guidance effect;
a routine for selecting at least one item and parameter from the items and dividing value candidates, on the basis of the feature quantity; and
a routine for creating a health guidance model associated with the order of priority in health guidance, using the items and parameters, as selected.

2. A health service support system according to claim 1, wherein the health guidance effect represents a medical care cost cutback effect per person in the case of guiding persons belonging to a category obtained by combining the items and dividing value candidates with each other, and the medical care cost cutback effect corresponds to an amount obtained when the persons belonging to the category obtained by combining the items and dividing value candidates with each other are divided into those having received guidance and those having received no guidance, and a medical care cost per person for those having received guidance is subtracted from a medical care cost per person for those having received no guidance.

3. A health service support system according to claim 1, further comprising a routine for displaying the health guidance effect and the feature quantity, wherein the routine for selecting the at least one item and parameter from the items and dividing value candidates, on the basis of the feature quantity, selects the at least one item and parameter against the health guidance effect and the feature quantity, as displayed.

4. A health service support system according to claim 1, wherein the routine for selecting the at least one item and parameter selects an item and parameter where a widening gap between the approximate straight lines corresponding the effect to the category having the maximum effect, and the effect of the category having the minimum effect, respectively, among the feature quantity for the at least one item and parameter, is the largest against a plurality of categories obtained by combining the items and dividing value candidates with each other.

5. A health service support system according to claim 3, wherein if a health guidance model previously created exists, the routine for setting the items and dividing value candidates adopts items and parameters used by the health guidance model previously created as one of the items and dividing value candidates, and the routine for displaying the health guidance effect and the feature quantity compares an health guidance effect in the case of using the items and parameters previously selected with an health guidance effect in the case of using the items and dividing value candidate newly adopted before displaying.

6. A health service support system according to claim 1, wherein the routine for computing the health guidance effect on the time-series basis make use of the health checkup data and the health guidance data during a time period “a”, and medical fee receipt data with the elapse of a time period “b” from the time period “a”, and computes a difference in the medical care cost between the time period “a” and a time period “c” due to existence or nonexistence of the guidance executed during the time period “a” as the guidance effect by the category obtained by combining items and parameters, associated with the health checkup data during the time period “a”, a change of the guidance effect, on the time-series basis, being found by repeating computation by shifting the time period “a” and the time period “c” by the time period “a” every time the computation is made.

7. A health service support system according to claim 1, further comprising an assessment and readjustment routine for computing an actual medical care cost on the basis of the health checkup data, the health guidance data, the medical fee receipt data, and the health guidance model, a forecast medical care cost on the basis of the health checkup data for a time period when the guidance is executed, and the health guidance model, and a planned medical care cost on the basis of the health checkup data when the health guidance model is created, and the health guidance model to be thereby compared with each other before displaying.

8. A health service support system according to claim 7, wherein the routine for creating the health guidance model creates a plurality of health guidance models, and the assessment and readjustment routine computes the planned medical care costs from the plurality of the health guidance models, respectively, so as to be displayed together with the actual medical care cost, and the forecast medical care cost.
